(19) United States
(12) Patent Application Publication
(10) Pub. No.: US 2017/0326094 A1
(43) Pub. Date: Nov. 16, 2017

(54) USES OF BIOACTIVE LIPIDS

(71) Applicant: NESTEC S.A., Vevey (CH)

(72) Inventors: Mojgan Masoodi, Lausanne (CH); El Hadji Mamadou Dioum, St-Prex (CH)

(21) Appl. No.: 15/529,058

(22) PCT Filed: Dec. 1, 2015
(86) PCT No.: PCT/EP2015/078218

§ 371 Date: May 23, 2017

(30) Foreign Application Priority Data
Dec. 9, 2014 (EP) 14197048.3

(51) Int. Cl. A61K 31/232; G01N 33/92; A61K 31/231; G01N 33/49; A61K 31/5575; G01N 33/00

(52) U.S. Cl. CPC A61K 31/232; G01N 33/49; A61K 31/5575; G01N 33/92; A61K 31/231; G01N 33/00; G01N 2800/7095

(57) ABSTRACT

The present invention provides an oxygenated fatty acyl glycerol for use in treating and/or preventing an inflammatory disease a subject.

### FIELD OF THE INVENTION

The present invention relates to treating and/or preventing an inflammatory disease. In particular the present invention relates to the use of oxygenated fatty acyl glycerol esters and methods utilising oxygenated fatty acyl glycerol esters for such treatment. The invention further relates to methods for determining this risk of a subject developing an inflammatory disease based on the level(s) of a oxygenated fatty acyl glycerol ester(s) in a sample from the subject.

### BACKGROUND TO THE INVENTION

Inflammation is the complex biological response of tissues to harmful stimuli, such as pathogens, damaged cells and/or irritants. It is generally a protective attempt by an organism to remove the injurious stimuli and to initiate the healing process for the tissue. However, non-appropriately regulated inflammation can lead to several diseases irrespective of the age of the subject.

Ageing is often associated with a dysregulation of the immune system, such as a noted decline in cell-mediated immune response concomitant with an increase humoral immune dysfunction, for example a lower response to a vaccine. Ageing is furthermore often associated with a state of low-grade inflammation. In particular many elderly subjects are at increased risk of infectious and non-infectious diseases that contribute to morbidity and mortality.

Unwanted inflammation can be treated by proper medication. However, medication may result in unwanted side effects and often requires the supervision of medical personnel.

Type 2 diabetes mellitus (TIID) is the most common form of diabetes and is characterized by chronic hyperglycemia, insulin resistance, and relative dysfunction of the pancreatic beta cells that normally secrete insulin in response to post prandial hyperglycemia. It is associated with genetic, environmental and behavioural risk factors.

People living with TIID are more vulnerable to various forms of both short- and long-term complications. Short-term complications include hypoglycaemia diabetic ketoacidosis (DKA), and hyperosmolar hyperglycaemic state (HHS). Long-term complications include retinopathy, cardiopathy, nephropathy and neuropathy. Such complications may lead to premature death.

This tendency of increased morbidity and mortality is observed in patients with TIID because of the prevalence of the disease, its insidious onset and late recognition. It is estimated that the global incidence of TIID was 366 million people in 2011 and that by 2030 this figure will have risen to 552 million (Global burden of diabetes. International Diabetes federation. Diabetic atlas fifth edition 2011, Brussels. Available at http://www.idf.org/diabetesatlas. (Accessed 18 Dec. 2011)).

A number of lifestyle factors are known to be associated with the development of TIID. These factors include physical inactivity, sedentary lifestyle, cigarette smoking and consumption of alcohol. In particular, obesity has been found to contribute to approximately 55% of cases of TIID (Morbidity and Mortality Weekly Report; 53(45): 1066-1068) and there is also a strong inheritable connection. However, it is recognised that not all obese individuals develop TIID.

TIID is characterized by insulin insensitivity as a result of insulin resistance, declining insulin production, and eventual pancreatic beta-cell failure. This leads to a decrease in glucose transport into the liver, muscle cells, and fat cells. As a result of this dysfunction, glucagon and hepatic glucose levels that rise during fasting are not suppressed with a meal. Given inadequate levels of insulin and increased insulin resistance, hyperglycemia results. An important feature of TIID is that pancreatic beta-cells become dysfunctional with an inability to sense nutrients as well as trophic factors and thus unresponsive to therapies which act specifically by increasing beta cell mass and levels of insulin secretion.

Current therapies for TIID include daily injection of glucagon-like peptide 1 (GLP1) receptor agonists to prevent beta cell loss and stimulate insulin secretion. However, use of GLP1 presents a risk of pancreatic and cardiovascular complications. More traditional oral drugs, such as sulfonyl urea, render patients prone to life threatening hypoglycaemia. There is also a lack of preventative therapies for prediabetics or high risk individuals and a lack of methods for identifying individuals who are at an increased risk of developing TIID.

There is thus the need for alternative compounds and compositions that can be used to treat and/or prevent inflammatory conditions and disorders.

### SUMMARY OF ASPECTS OF THE INVENTION

The present invention is based on the determination that oxygenated fatty acyl glycerol ester levels are associated with inflammatory disease. Further, the present invention has demonstrated that oxygenated fatty acyl glycerol esters can influence physiological responses in cells which are directly relevant to such inflammatory diseases.

In a first aspect, the present invention provides an oxygenated fatty acyl glycerol ester for use in treating and/or preventing an inflammatory disease a subject.

The oxygenated fatty acyl glycerol ester may be an oxygenated arachidonyl glycerol ester. The oxygenated fatty acyl glycerol ester may be a prostaglandin glycerol ester. The oxygenated fatty acyl glycerol ester may be a prostatetraenoic acid glycerol ester.

The prostatetraenoic acid glycerol ester may be selected from the following group: 11-oxo-5Z,9,12E,14E-prostatetraenoic acid-1 glycerol ester; 9, 15S-dihydroxy-11-oxo-5Z,13E-prostadienoic acid, 1-glyceryl ester; 11-oxo-5Z,9,12E,4E-prostatetraenoic acid-2-glycerol ester; 11-oxo-15S-hydroxy-5Z,9Z,13E-prostatrienoic acid-1 glycerol ester; and 9, 15S-dihydroxy-11-oxo-5Z,13E-prostadienoic acid, 2-glyceryl ester.

The prostatetraenoic acid glycerol ester may be 11-oxo-5Z,9,12E,14E-prostatetraenoic acid-1 glycerol ester, 9, 15S-dihydroxy-11-oxo-5Z,13E-prostadienoic acid, 1-glyceryl ester or 11-oxo-5Z,9,12E,14E-prostatetraenoic acid-2-glycerol ester.

In a second aspect, the present invention provides a composition comprising one or more oxygenated fatty acyl glycerol esters as defined in the first aspect of the invention for use in treating and/or preventing an inflammatory disease in a subject.

The inflammatory disease may be selected from the following group: Type II diabetes, insulin resistance, obesity and metabolic diseases.

The oxygenated fatty acyl glycerol ester or composition for use according to the first or second aspect of the invention may be for preventing or delaying the onset of Type II diabetes in an obese subject.

The oxygenated fatty acyl glycerol ester or composition for use according to the first or second aspect of the invention may be for modulating insulin secretion in a subject.

The oxygenated fatty acyl glycerol ester may act on a cell selected from the following group: a pancreatic cell, an enteroendocrine cell, an epithelial cell, a liver cell, an adipocyte, or a neural cell.

The cell may be a pancreatic beta cell. The oxygenated fatty acyl glycerol ester may increase the level of insulin produced by the pancreatic beta cell. The oxygenated fatty acyl glycerol ester may prevent or reduce apoptosis of pancreatic beta cells.

The cell may be an enteroendocrine L cell.

The cell may be an astrocyte or a neuron.

The oxygenated fatty acyl glycerol ester may reduce inflammation in liver and/or adipose tissues.

In a third aspect the present invention provides a method for inducing or increasing production of at least one oxygenated fatty acyl glycerol ester as defined in the first aspect of the invention in vivo.

The oxygenated fatty acyl glycerol ester level may be increased in a liver cell, white adipose tissue or a pancreatic beta cell.

The method may comprise the step of:

(a) administering a precursor selected from the following group; arachidonyl glyercol (AG), diacylglycerol (1,2-DAG) and/or triacylglycerol (TAG) to a subject.
(b) inducing or increasing the expression or activity of an enzyme selected from the following group Phospholipase C (PLC), Diacylglycerol lipase (DAGL), Phospholipase A2 (PLA₂), N-acetyltransferase 2 (NAT), N-acyl phosphatidylethanolamine-specific phospholipase D (NATE-PLD), Cyclooxygenase-2 (COX-2), prostaglandin F synthase (PGFS), prostaglandin E synthase (PROSTAGLANDIN GLYCEROL ESTER S), prostaglandin I synthase (PGIS), prostaglandin D synthase (PGDS) and/or thromboxane A(2) synthase (TXAS) in a subject.

In a fourth aspect, the present invention provides a method for treating and/or preventing an inflammatory disease in a subject which comprises the step of administering a oxygenated fatty acyl glycerol ester as defined in the first aspect of the invention to a subject or inducing or increasing production of at least one oxygenated fatty acyl glycerol ester as defined in the first aspect of invention in vivo by a method according to the third aspect of the invention.

The inflammatory disease may be selected from the following group: Type II diabetes, insulin resistance, obesity and metabolic diseases.

The inflammatory disease may be Type II diabetes.

The method according to the fourth aspect of the invention may be for preventing or delaying the onset of Type II diabetes in an obese subject.

The method may be for modulating insulin secretion in a subject.

In a fifth aspect, the present invention provides a method for identifying a subject at risk of developing an inflammatory disease, comprising:

(a) determining a level of at least one oxygenated fatty acyl glycerol ester in a sample from the subject,
(b) comparing the level(s) of the oxygenated fatty acyl glycerol ester(s) in the sample to reference values;

wherein a lower level(s) of the oxygenated fatty acyl glycerol ester(s) in the sample compared to the reference levels is indicative of the risk of developing an inflammatory disease.

The method for identifying a subject at risk of developing an inflammatory disease may be followed by administration of a dietary intervention to increase oxygenated fatty acyl glycerol esters.

The oxygenated fatty acyl glycerol ester may be an oxygenated arachidonyl glycerol ester. The oxygenated fatty acyl glycerol ester may be a prostaglandin glycerol ester. The oxygenated fatty acyl glycerol ester may be a prostatetraenoic acid glycerol ester.

The prostatetraenoic acid glycerol ester may be selected the following group:
11-oxo-5Z,9,12E,14E-prostatetraenoic acid-1 glycerol ester;
9, 15S-dihydroxy-11-oxo-5Z,13E-prostadienoic acid, 1-glyceryl ester;
11-oxo-5Z,9,12E,4E-prostatetraenoic acid-2-glycerol ester;
11-oxo-15S-hydroxy-5Z,9Z,13E-prostatrienoic acid-1 glycerol ester; or
9, 15S-dihydroxy-11-oxo-5Z,13E-prostadienoic acid, 2-glyceryl ester.

The sample may be a serum, plasma, urine or adipose tissue biopsy sample.

The inflammatory disease may be selected from the following group of: Type II diabetes, insulin resistance, obesity and metabolic diseases.

In one embodiment, the subject is obese and the method is used to predict the likelihood of the subject developing Type II diabetes.

In a sixth aspect, the present invention provides a oxygenated fatty acyl glycerol ester as defined in the first aspect of the invention for use in
i) regulating inflammatory cytokine signalling in a cell; or
ii) protecting a cell against apoptosis.

### DESCRIPTION OF THE FIGURES

FIG. 1. Concentration of stock and various dilution of the bioactive lipid fractions isolated from activated WAT (white adipose tissues) in ethanol. Synergistic effect of bioactive lipids on glucose stimulated insulin secretion. MIN6 cells are stimulated with 20 mM glucose together with lipid fractions (1:50 dilution) or vehicle (Ethanol 2%) for 30 minutes after starvation in 2 mM glucose for 2 hrs. The concentration of the respective bioactive lipid fractions is mentioned below. Secreted insulin was measured by ELISA.

FIG. 2—Bioactive lipid fraction dose response and pancreatric beta cell survival . . . MIN6 cells (70-80% confluent) were treated with various dilutions of the isolated bioactive lipids (1:1000 to 1:20 dilution) in complete DMEM medium for 48 hrs ( [symbol] second, lighter box) or with the corresponding dilution of Ethanol, the vehicle control ( [symbol] first, darker box). Attached cells were trypsinized and counted. The concentration of the various dilutions is shown in FIG. 1.

FIG. 3—Long-term effect of bioactive lipids on beta cell function. (A) MIN6 cells were treated with bioactive lipids at a concentration close to physiological ranges (1:1000 dilution) for 72 hours. At the end of the treatment, beta cell function was assessed by measuring GSIS. (B) Bioactive fractions 3 and 5 were tested in primary human islets from a healthy donor for 72 hrs. Bioactive lipid fraction 5 substantially improved beta cell function by doubling the capacity of the human islets beta cells to secrete insulin in response to glucose stimulation.

FIG. 4—Bioactive lipid acutely amplify glucose stimulated insulin secretion (GSIS). Insulin secretion was measured in MIN6 cells under starving condition (2 mM glucose) or after stimulation with 20 mM glucose or 20 mM glucose plus bioactive lipids at a 1:100 dilution for 15 minutes. Insulin secretion was measured by ELISA.

FIG. 5—Bioactive lipid fraction 5 is further separated into 5 sub-fractions (5-, 5.1, 5.2, 5.3, and 5.4) MIN6 cells were treated with the enriched bioactive lipid sub-fractions for 72 hours in a 1:1000 dilution before performing GSIS.

FIG. 6—Beta cells were treated with an inflammatory cytokine cocktail (50 U/mL IL1β, 100 U/mL TNFα and 100 U/mL INFγ) for 48 hrs in the presence or absence of bioactive lipid fractions (1:100 dilution). After treatment, NFkB signaling pathway (IKKa/b phosphorylation) and apoptosis (cleaved caspase 3) were assessed by Western blot (A), also Caspase 8 activity was measured in cell extracts (B) using the Caspase Glo kit (Promega).

FIG. 7—Isolated islet cells from WT Wistar rats or from Gata Kakizaki (GK) rats were treated with a bioactive lipid fraction for 72 hours. To measure beta cell function, Islet cells were then stimulated with a nutrient cocktail (20 mM glucose, lx amino acid and 0.1 μM Ex-4) for 1 hr and insulin secretion was assessed by ELISA.

FIG. 8—Acute stimulation of the enteroendocrine L cell line (NCI-H716) was tested with low (2 mM) and high (20 mM) glucose in the presence or absence of bioactive lipid fraction. The effect was assessed by measuring GLP1 secretion.

FIG. 9—Long-term effect of bioactive lipids in enteroendocrine L cell function was determined by pretreating the NCI-H716 cell line with the bioactive lipids for 72 hrs before assessing GLP1 secretion after glucose stimulation.

FIG. 10—Regulation of cellular stress genes in MIN6 cells after treatment with bioactive lipid fractions for 72 hrs

FIG. 11—Workflow for identification of bioactive lipids

FIG. 12—Comparison of the functional effects of isolated Fraction 5.4 with synthetic pure fractions 5.4 and 5.3. Insulin secretion was assessed after acute (1 hour) and chronic (72 hours) treatment with bioactive lipids. (a) Human islet cells. (b) Primary young rat islet cells. (c) INS1E p81 and INSE p96

FIG. 13—Glucose stimulated insulin secretion with bioactive lipid

The bioactive lipid prostaglandin D2 glycerol ester (PGD2G) identified from fraction 5.3 increased insulin secretion in mouse islets (A) or Ins1E cells (B) after treatment for 72 hours at 50 μM. After PGD2G treatment, glucose stimulated insulin release was measured in low glucose (2 mM) and high glucose (20 mM) conditions in KRB solution. The insulin release is expressed as released from the total content of insulin in Ins1E cells and mouse islets.

FIG. 14—Insulin secretion with bioactive lipid normalised to total protein content prostaglandin D2 glycerol ester (PGD2G) identified from fraction 5.3 acutely stimulated insulin secretion upon stimulation with glucose. Glucose stimulated insulin release was measured in low glucose (2 mM) and high glucose (20 mM) in the presence of various concentrations (470 μM, 2.3 nM, 230 nM) of the bioactive lipid. The bioactive lipid improved glucose stimulated insulin release particularly at concentrations from 2.5 nM to 250 nM. The result are presented as insulin release normalized to total protein content.

FIG. 15—Improvement of Beta Cell Function and Incretin Response in Human Islets with Bioactive Lipid

The bioactive lipid, prostaglandin D2 glycerol ester (PGD2G) identified from fraction 5.3 at 50 μM improved beta cell function and the incretin response in human islets from donors: (A) lean type 2 diabetic patient and (B) a non-diabetic obese after treatment for 72 hours. Glucose stimulated insulin release was measured in low glucose (2 mM), high glucose (20 mM) and high glucose (20 mM)+0.1 uM Exendin4

FIG. 16—Improvement of glucose stimulated insulin release after cytokine-induced dysfunction

Bioactive lipid prostaglandin D2 glycerol ester (PGD2G) identified from fraction 5.3 PGD2G protected human islets against cytokine induced dysfunction. Human islets from: (A) a lean non-diabetic donor, (B) a lean type 2 diabetic donor and (C) an obese type 2 diabetic donor were treated for 72 hours with the bioactive lipid at 50 pM. During the last 48 hours, the cytokine mix was added (IL1beta 10 ng/ml, TNF alpha 25 ng/ml and INFgamma 10 ng/ml). Glucose stimulated insulin release was measured in low glucose (2 mM) and high glucose (20 mM) conditions. The bioactive lipid was able to improve glucose stimulated insulin release after cytokine-induced dysfunction.

FIG. 17—Bioactive lipid increases GLP-1 secretion

Bioactive lipid prostaglandin D2 glycerol ester (PGD2G) identified from fraction 5.3 increased GLP1 secretion. GLP-1 secretion assay was performed using human H716 cells in the presence of various concentration of the bioactive lipid from 0.23 nM to 2.3 nM. Prostaglandin D2 glycerol ester significantly improved GLP1 secretion in H716 cells (expressed as GLP1 release normalized to total protein content).

FIG. 18—Bioactive lipids 15-deoxy-Δ12,14-PGJ2-2-G identified from fraction 5.4 and prostaglandin D2 glycerol ester (PGD2G) identified from fraction 5.3 increase insulin secretion

Insulin secretion assay was performed in low glucose (2 mM) and high glucose (20 mM) conditions with human islets from a lean non-diabetic donor. Bioactive lipid, 15-deoxy-Δ12,14-PGJ2-2-G identified from fraction 5.4 (250 pM) increased insulin secretion both with and without the presence of a white adipose tissue (WAT) fraction ( 1/100 dilution). Bioactive lipid, prostaglandin D2 glycerol ester (PGD2G) identified from fraction 5.3 also increased insulin secretion compared to control tissues. Both bioactive lipids improved glucose stimulated insulin release acutely. The results are expressed as ng secreted insulin per 10 islets.

### DETAILED DESCRIPTION

In one aspect the present invention provides to an oxygenated fatty acyl glycerol ester for use in treating and/or preventing an inflammatory disease in a subject.

### Oxygenated Fatty Acyl Glycerol Ester

An oxygenated fatty acyl glycerol ester may also be referred to herein as a “bioactive lipid”.

An oxygenated fatty acyl glycerol ester refers to a bioactive lipid which comprises glycerol bonded to at least one oxygenated fatty acid moiety, or a derivative thereof, by an ester linkage. The oxygenated fatty acyl glycerol ester may comprise one, two or three oxygenated fatty acid moieties, or a derivative thereof, bonded by an ester linkage to any carbon in the glycerol moiety.

For example, an oxygenated fatty acyl glycerol ester may have the following structure:

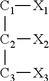

wherein at least one of X₁, X₂ and X₃ is an oxygenated fatty acid bonded to the carbon by an ester linkage.

A ‘fatty acid moiety’ refers to a carboxylic acid with a long aliphatic tail. The fatty acid moiety may comprise 4 to 28 carbon atoms. The fatty acid moiety may be saturated or unsaturated. Short chain fatty acids have fewer than six carbons, medium chain fatty acids have 6-12 carbons, long chain fatty acids have 13 to 21 carbons and very long chain fatty acids have more than 22 carbons.

The fatty acid may be a long chain fatty acid or a very long chain fatty acid.

Examples of fatty acids include, but are not limited to, arachidonic acid, myristoleic acid, palmitoleic acid, sapienic acid, oleic acid, elaidic acid, vaccenic acid, linoleic acid and eicosapentaenoic acid

‘Oxygenated’ means that the fatty acid moiety comprises at least one oxygenated functional group within the fatty acid chain. That is, it comprises at least one oxygenated functional group in addition to the ester group connecting it to the glycerol moiety.

The oxygenated functional group may be, for example, a hydroxyl, epoxy, methoxy or oxo functional group. In certain embodiments the oxygenated functional group is a hydroxyl group.

‘A derivative thereof’ refers to any molecule which can be formed from the oxygenated fatty acid molecule. For example, a derivative thereof may refer to an oxygenated arachidonyl, a prostaglandin or a prostatetraeonic acid moiety.

### Oxygenated Arachidonyl Glycerol Ester

An oxygenated arachidonyl glyercol ester refers to a glyercol ester in which at least one oxygenated arachidonic acid moiety is linked to the glycerol moiety by an ester linkage.

The oxygenated arachidonyl glyercol ester may comprise one, two or three arachidonic acid groups linked to the glycerol moiety via an ester linkage. The oxygenated arachidonyl glyercol ester may comprise a single arachidonic acid group linked to the glycerol moiety via an ester linkage. The single arachidonic acid group may be linked via an ester linkage to C₁, C₂ or C₃ of the glycerol moiety.

### Prostaglandin Glycerol Ester

A prostaglandin glycerol ester refers to a glycerol ester in which at least one prostaglandin moiety is linked to the glycerol moiety by an ester linkage.

Prostaglandin glycerol esters are mainly generated by the oxygenation of 2-arachidonyl glycerol via cyclooxygenase, other specific enzymes such as prostaglandin D/E synthases are also involved in synthesis of specific prostaglandin glycerols. Prostaglandins are derived enzymatically from fatty acyls and contains 20 carbon atoms, including a 5-carbon ring.

Examples of prostaglandins include, but are not limited to, prostaglandin A2 (PGA2), PGB2, PGC2, PGD2, PGE2 (PGE2), PGF2a and PGG2.

The prostaglandin glycerol ester may comprise one, two or three prostaglandin moieties linked to the glycerol moiety via an ester linkage. The prostaglandin glyercol ester may comprise a single prostaglandin group linked to the glycerol moiety via an ester linkage. The single prostaglandin group may be linked via an ester linkage to C₁, C₂ or C₃ of the glycerol moiety.

### Prostatetraenoic Acid Glycerol Ester

A prostatetraenoic acid glycerol ester refers to a glycerol ester in which at least one prostatetraenoic acid moiety is linked to the glycerol moiety by an ester linkage.

Prostatetraenoic acid glycerol esters are mainly generated by the oxygenation of 2-arachidonyl glycerol via cyclooxygenase,

The prostatetraenoic acid glycerol ester for use according to the present invention may be selected from the following group: 11-oxo-5Z,9,12E,14E-prostatetraenoic acid-1 glycerol ester; 9, 15S-dihydroxy-11-oxo-5Z,13E-prostadienoic acid, 1-glyceryl ester; 11-oxo-5Z,9,12E,4E-prostatetraenoic acid-2-glycerol ester; 11-oxo-15S-hydroxy-5Z,9Z,13E-prostatrienoic acid-1 glycerol ester; and 9, 15S-dihydroxy-11-oxo-5Z,13E-prostadienoic acid, 2-glyceryl ester.

The prostatetraenoic acid glycerol ester may be 11-oxo-5Z,9,12E,14E-prostatetraenoic acid-1 glycerol ester, 9, 15S-dihydroxy-11-oxo-5Z,13E-prostadienoic acid, 1-glyceryl ester or 11-oxo-5Z,9,12E,14E-prostatetraenoic acid-2-glycerol ester.

### Composition

In one aspect the present invention relates to a composition comprising one or more oxygenated fatty acyl glycerol esters as described herein.

The composition may comprise at least one, at least two, at least three, at least four or at least five oxygenated fatty acyl glycerol esters.

The composition may comprise one or more prostatetraenoic acid glycerol esters selected from the following group: 11-oxo-5Z,9,12E,14E-prostatetraenoic acid-1 glycerol ester; 9, 15S-dihydroxy-11-oxo-5Z,13E-prostadienoic acid, 1-glyceryl ester; 11-oxo-5Z,9,12E,4E-prostatetraenoic acid-2-glycerol ester; 11-oxo-15S-hydroxy-5Z,9Z,13E-prostatrienoic acid-1 glycerol ester; and 9, 15S-dihydroxy-11-oxo-5Z,13E-prostadienoic acid, 2-glyceryl ester.

### Pharmaceutical Composition

The oxygenated fatty acyl glycerol ester or composition for use according to the present invention may be provided as a pharmaceutical composition.

The pharmaceutical composition may comprise one or more oxygenated fatty acyl glycerol esters as defined herein along with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents.

### Administration

The administration of the oxygenated fatty acyl glycerol ester can be accomplished using any route that makes the active ingredient bioavailable. For example, the oxygenated fatty acyl glycerol ester can be administered by oral and parenteral routes, intraperitoneally, intravenously, subcutaneously, transcutaneously, intramuscularly, via local delivery for example by catheter or stent.

Treating and/or Preventing

The present invention provides a oxygenated fatty acyl glycerol ester for use in treating and/or preventing an inflammatory disease in a subject.

The use for the prevention of an inflammatory disease relates to the prophylactic use of the oxygenated fatty acyl glycerol ester. Herein the oxygenated fatty acyl glycerol ester may be administered to a subject who has not yet contracted an inflammatory disease and/or who is not showing any symptoms of the disease to prevent or impair the cause of the disease or to reduce or prevent development of at least one symptom associated with the disease. The subject may have a predisposition for, or be thought to be at risk of developing, an inflammatory disease.

The use for the treatment of an inflammatory disease relates to the therapeutic use of the oxygenated fatty acyl glycerol ester. Herein the oxygenated fatty acyl glycerol ester may be administered to a subject having an existing disease or condition in order to lessen, reduce or improve at least one symptom associated with the disease and/or to slow down, reduce or block the progression of the inflammatory disease.

### Subject

The subject may be a human or animal subject. The subject may be a mammalian subject. In one embodiment, the subject is a mammal, preferably a human. The subject may alternatively be a non-human mammal, including for example a horse, cow, sheep or pig. In one embodiment, the subject is a companion animal such as a dog or cat.

The subject may have an inflammatory disease, as described herein. ‘Having an inflammatory disease’ refers to a subject having at least one symptom associated with the condition.

The subject may be at risk of an inflammatory disease, as described herein. ‘At risk of an inflammatory disease’ refers to a subject who has not yet contracted an inflammatory disease and/or who is not showing any symptoms of the disease. The subject may have a predisposition for, or be thought to be at risk of developing, an inflammatory disease.

### Inflammatory Disease

In one aspect the present invention provides a oxygenated fatty acyl glycerol ester for use in treating and/or preventing an inflammatory disease. Typical inflammatory diseases are known to those of skill in the art and include, but are not limited to, diseases including cardiovascular disease, cancer, arthritis, autoimmune-related conditions, obesity, metabolic diseases, insulin resistance and Type II diabetes mellitus.

Inflammation is the complex biological response of tissues to harmful stimuli, such as pathogens, damaged cells and/or irritants. It is generally a protective attempt by an organism to remove the injurious stimuli and to initiate the healing process for the tissue. However, non-appropriately regulated inflammation can lead to several diseases irrespective of the age of the subject.

The inflammatory disease may be associated with ageing.

Ageing is often associated with a dysregulation of the immune system, such as a noted decline in cell-mediated immune response concomitant with an increase humoral immune dysfunction, for example a lower response to a vaccine. Ageing is furthermore often associated with a state of low-grade inflammation. In particular many elderly subjects are at increased risk of infectious and non-infectious diseases that contribute to morbidity and mortality.

### Obesity

Obesity is caused by an excessive accumulation of white adipose tissue (WAT). It is associated with severe metabolic disorders (metabolic syndrome, MS) and represents one of the key problems of health care systems in affluent societies.

“Body mass index” or “BMI” means the ratio of weight in kg divided by the height in metres, squared. “Overweight” is defined for an adult human as having a BMI between 25 and 30. “Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30.

WAT generates a number of signals, which include cytokines, hormones, growth factors, complement factors and matrix proteins that not only affect the neighbouring cells but also target other peripheral tissues as well as the brain. A systemic inflammatory process, including activation of the innate immune system, is triggered by adipose tissue expansion and hypoxia.

Thus obesity is associated with chronic low-grade inflammation of WAT which, in turn, may affect metabolism of adipocytes. This chronic inflammation is associated with various inflammatory markers including, but not limited to, IL-6, IL-8, IL-18, TNF-α and C-reactive protein.

Obesity-associated chronic low-grade inflammation is an important cause of obesity-induced insulin resistance and is a risk factor for the development of type 2 diabetes mellitus (TIID). Although obesity is one of the major risk factors for TIID, not all obese subjects become diabetic. Obesity-associated chronic low-grade inflammation is also recognized as an important cause of obesity-induced insulin resistance.

Thus the subject may be an obese subject at risk of developing insulin resistance and/or TIID.

### Insulin Resistance

Insulin resistance may be defined as a reduced responsiveness of a target cell or a whole organism to the insulin concentration to which it is exposed. This definition is generally used to refer to impaired sensitivity to insulin mediated glucose disposal.

Insulin is the pivotal hormone regulating cellular energy supply and macronutrient balance, directing anabolic processes of the fed state. It is essential for the intracellular transport of glucose to insulin-dependent tissues such as muscle and adipose tissue. Physiologically, at the whole body level, the actions of insulin are influenced by the interplay of other hormones. Insulin, though the dominant hormone driving metabolic processes in the fed state, acts in concert with growth hormone and insulin-like growth factor 1 (IGF-1); growth hormone is secreted in response to insulin, among other stimuli, preventing insulin-induced hypoglycaemia. Other counter-regulatory hormones include glucagon, glucocorticoids and catecholamines. These hormones drive metabolic processes in the fasting state.

Insulin resistance may manifest at the cellular level via post-receptor defects in insulin signalling. Possible mechanisms include down-regulation, deficiencies or genetic polymorphisms of tyrosine phosphorylation of the insulin receptor, IRS proteins or PIP-3 kinase, or may involve abnormalities of GLUT 4 function (Wheatcroft et al; Diabet Med. 2003; 20:255-68).

Insulin resistance correlates with increasing body mass index, waist circumference and in particular waist-hip ratio. These reflect increased adiposity especially increased levels of visceral adipose tissue. Visceral adipose tissue refers to intra-abdominal fat around the intestines and correlates with liver fat. Visceral adipose tissue has metabolic characteristics which differ from that of subcutaneous fat. It is more metabolically active with regard to free fatty acyl turnover; the increased flux of free fatty acyls promotes insulin resistance at a cellular level and increases hepatic VLDL production.

Adipose tissue produces a number of cytokines which have been associated with insulin resistance, including those with pro-inflammatory activity e.g. TNFα, interleukins, and PAI-1.

The insulin resistance seen in obesity is believed to primarily involve muscle and liver, with increased adipocyte-derived free fatty acyls promoting triglyceride accumulation in these tissues. This is more likely where adipocytes are insulin resistant. Free fatty acyl flux is greater from visceral adipose tissue and more likely in those individuals with genetically mediated adipocyte insulin resistance. Whilst individual differences in the effects of increasing adiposity exist, weight gain worsens and weight loss improves insulin resistance in those so predisposed.

Thus the insulin resistance may be obesity-induced insulin resistance.

The subject may be an insulin resistant subject at risk of developing TIID.

### Type II Diabetes Mellitus (TIID)

TIID is a chronic metabolic disorder which is increasing in prevalence globally. In some countries of the world the number of people affected is expected to double in the next decade due to an increase in the ageing population.

TIID is characterized by insulin insensitivity as a result of insulin resistance, declining insulin production, and eventual pancreatic beta-cell failure. This leads to a decrease in glucose transport into the liver, muscle cells, and fat cells. There is an increase in the breakdown of fat associated with hyperglycemia.

As a result of this dysfunction, glucagon and hepatic glucose levels that rise during fasting are not suppressed with a meal. Given inadequate levels of insulin and increased insulin resistance, hyperglycemia results.

People with TIID are more vulnerable to various forms of both short- and long-term complications, including diabetic ketoacidosis (DKA), hyperosmolar hyperglycaemic state (HHS), retinopathy, cardiopathy, nephropathy and neuropathy. These complications may lead to premature death.

The present inventors have surprisingly shown that oxygenated fatty acyl glycerol esters can increase insulin secretion from pancreatic beta cells and reduce levels of apoptosis in pancreatic beta cells.

Thus in one aspect the present invention provides a oxygenated fatty acyl glycerol ester for use in modulating insulin secretion in a subject. Modulating insulin secretion may refer to increasing levels of insulin secretion in a subject. For example, the oxygenated fatty acyl glycerol ester may cause an increase in the level of insulin secretion by 1.5-, 2-, 5- or 10-fold compared to the level in an equivalent untreated control.

An important feature of TIID is that pancreatic beta-cells become dysfunctional, insensitive to glucose stimulationand thus unresponsive to therapies which act specifically by increasing levels of insulin secretion. The oxygenated fatty acyl glycerol esters for use as described herein act through a range of functions, including modulating general inflammation, mitochondrial function and apoptosis. Thus the present oxygenated fatty acyl glycerol esters are advantageous as a therapy for TIID as they positively modulate mechanisms and pathways which are known to contribute to the development of insulin resistance in TIID, in addition to stimulating insulin secretion.

As described above, obesity is a major risk factor for the development of TIID, however, not all obese patients go on to develop TIID.

Thus, in one aspect present invention provides a oxygenated fatty acyl glycerol ester for use in preventing or delaying the onset of TIID in an obese subject.

### Metabolic Diseases

A metabolic disease or disorder is a condition characterised by an alteration or disturbance in metabolic function. Metabolic disorders include but are not limited to hyperglycemia, prediabetes, diabetes (type I and type II), obesity, insulin resistance and metabolic syndrome.

### Lipodystrophy

The oxygenated fatty acyl glycerol ester of the invention may be used for treating and/or preventing lipodystrophy, which is a medical condition characterized by abnormal or degenerative conditions of the body's adipose tissue. In particular lipodystrophy can be a lump or small dent in the skin that forms when a person performs insulin injections repeatedly in the same spot.

One of the side-effects of lipodystrophy is the rejection of the injected medication, the slowing down of the absorption of the medication, or trauma that can cause bleeding that, in turn, will reject the medication. In any of these scenarios, the dosage of the medication, such as insulin for diabetics, becomes impossible to gauge correctly and the treatment of the disease for which the medication is administered is impaired, thereby allowing the medical condition to worsen.

### Cell

The oxygenated fatty acyl glycerol ester for use according to the present invention may act on cell selected from the following group: a pancreatic cell, an enteroendocrine cell, an epithelial cell, a liver cell, an adipocyte, or a neural cell.

The term ‘act on’, as used herein, means to cause a change in the physiological activities of the cell.

The oxygenated fatty acyl glycerol ester may, for example, stimulate secretion of a hormone such as insulin, glucagon-like peptide-1 (GLP1) and/or gastric inhibitory polypeptide (GIP) by the cell. The oxygenated fatty acyl glycerol ester may prevent apoptosis of the cell, in particular apoptosis associated with oxidative or inflammatory stress. The oxygenated fatty acyl glycerol ester may rescue the insulin secretion capacity of the cell.

The cell may be sensitive to oxidative and/or inflammatory stress.

The cell may be involved in the regulation of lipid metabolism.

Enteroendocrine cells are specialized endocrine cells of the gastrointestinal tract and pancreas. They produce hormones in response to various stimuli gastrointestinal hormones or peptides and release them into the bloodstream for systemic effect, diffuse them as local messengers, or transmit them to the enteric nervous system to activate nervous responses.

The pancreas is an endocrine gland producing several important hormones, including insulin, glucagon, somatostatin, and pancreatic polypeptide which circulate in the blood. The islets of Langerhans are the regions of the pancreas that contain its endocrine (i.e., hormone-producing) cells. Hormones produced in the islets of Langerhans are secreted directly into the blood flow by (at least) five types of cells as follows:

Alpha cells producing glucagon (15-20% of total islet cells)

Beta cells producing insulin and amylin (65-80%)

Delta cells producing somatostatin (3-10%)

PP cells (gamma cells) producing pancreatic polypeptide (3-5%)

Epsilon cells producing ghrelin (<1%).

The oxygenated fatty acyl glycerol ester for use according to the present invention may act on a pancreatic beta cell. Pancreatic beta cells are the insulin producing cells of the pancreas and are the most abundant cells in the islet of Langerhans.

Endocrine cells secrete hormones. They may, for example, be intestinal, gastric or pancreatic endocrine cells.

Intestinal endocrine cells are not clustered together but spread as single cells throughout the intestinal tract. Hormones secreted include somatostatin, motilin, cholecystokinin, neurotensin, vasoactive intestinal peptide, and enteroglucagon.

The oxygenated fatty acyl glycerol ester for use according to the present invention may act on a K cell or an L cell. K cells secrete gastric inhibitory peptide, an incretin. L cells secrete glucagon-like peptide-1, also an incretin, and glucagon-like peptide-2.

Enterochromaffin cells are endocrine cells secreting serotonin and histamine.

Gastric endocrine cells are found at stomach glands, mostly at their base. The G cells secrete gastrin, post-ganglionic fibers of the vagus nerve can release gastrin-releasing peptide during parasympathetic stimulation to stimulate secretion.

Other hormones produced by gastric endocrine cells include cholecystokinin, somatostatin, vasoactive intestinal peptide, substance P, alpha and gamma-endorphin.

Epithelial cells cover the inner and outer linings of body cavities, such as the stomach and the urinary tract. Some epithelial cells, such as the ones found on the intestinal lining, aid in the transportation of filtered material through the use active-transport systems located on the apical side of their plasma membranes. For example, the glucose-Na+ symports located within certain domains of the plasma membrane of epithelial cells lining the intestine enable the cells to generate Na+ concentration gradients across their plasma membranes, which provides the energy needed to uptake glucose, from the lumen of the intestine. The glucose is then released into the underlying connective tissues and is transported into the blood supply through facilitated diffusion down its concentration gradient.

The cell may be a liver cell such as a hepatocyte. The liver is involved in carbohydrate metabolism as it forms fatty acyls from carbohydrates and synthesizes triglycerides from fatty acyls and glycerol. Hepatocytes also synthesize apoproteins with which they then assemble and export lipoproteins (VLDL, HDL). The liver is also the main site in the body for gluconeogenesis, the formation of carbohydrates from precursors such as alanine, glycerol, and oxaloacetate.

The liver is also involved in lipid metabolism as it receives many lipids from the systemic circulation and metabolizes chylomicron remnants. It also synthesizes cholesterol from acetate and further synthesizes bile salts.

Adipocytes are the cells that primarily compose adipose tissue, specialized in storing energy as fat. There are two types of adipose tissue, white adipose tissue (WAT) and brown adipose tissue (BAT), which are also known as white fat and brown fat, respectively, and comprise two types of fat cells. Obesity is characterized by the expansion of fat mass, through adipocyte size increase (hypertrophy) and, to a lesser extent, cell proliferation (hyperplasia). In the fat cells of obese individuals, there is increased production of metabolism modulators, such as glycerol, hormones, and pro-inflammatory cytokines, leading to the development of insulin resistance.

Fat production in adipocytes is strongly stimulated by insulin which promotes unsaturated fatty acyl synthesis, glucose uptake and activates the transcription of genes that stimulate lipogenesis.

The cell may be a neural cell such as a neuron or an astrocyte. Astrocytes are star-shaped glial cells in the brain and spinal cord. They are the most abundant cells of the human brain. They perform many functions, including biochemical support of endothelial cells that form the blood-brain barrier, provision of nutrients to the nervous tissue, maintenance of extracellular ion balance, and a role in the repair and scarring process of the brain and spinal cord following traumatic injuries.

### Method

The present invention further relates to a method for inducing or increasing production of at least one oxygenated fatty acyl glycerol ester as defined in the first aspect of the invention in vivo.

The method may induce or increase the production of at least one, at least two, at least three, at least four, up to a plurality of oxygenated fatty acyl glycerol esters as defined in the first aspect of the invention.

The method may cause an increase in the level of the oxygenated fatty acyl glycerol ester in the liver and/or the white adipose tissue of the subject. The term increase may refer, for example, to a 1.5-, 2-, 5-, or 10-fold increase in the level of the oxygenated fatty acyl glycerol ester compared the level before the method was performed. The oxygenated fatty acyl glycerol esters may not be present in the liver and/or the white adipose tissue of the subject prior to the method being performed.

The method may comprise the step of:

a) administering a precursor selected from the group of arachidonyl glyercol (AG), diacylglycerol (1,2-DAG) and/or triacylglycerol (TAG) to a subject and/or
(b) inducing or increasing the expression or activity of an enzyme selected from the following group Phospholipase C (PLC), Diacylglycerol lipase (DAGL), Phospholipase A2 (PLA2), N-acetyltransferase 2 (NAT), N-acyl phosphatidylethanolamine-specific phospholipase D (NATE-PLD), Cyclooxygenase-2 (COX-2), prostaglandin F synthase (PGFS), prostaglandin E synthase (PGES), prostaglandin I synthase (PGIS), prostaglandin D synthase (PGDS) and/or thromboxane A(2) synthase (TXAS) in a subject.

The expression of an enzyme as described above may be increased by gene therapy, stimulating an immune response, local infiltration of immune cells or alteration in lipid pools and/or lipid rafts.

The administration of the precursor may be accomplished using any of a variety of routes that make the active ingredient bioavailable. For example, the precursor can be administered by oral and parenteral routes, intraperitoneally, intravenously, subcutaneously, transcutaneously or intramuscularly, via local delivery.

The present invention also provides a oxygenated fatty acyl glycerol ester precursor for use in treating and/or preventing an inflammatory disease.

### Method of Treatment

The present invention further relates to a method for treating and/or preventing an inflammatory disease in a subject which comprises the step of administering at least one oxygenated fatty acyl glycerol ester as defined in the first aspect of the invention to a subject or inducing or increasing production of at least one oxygenated fatty acyl glycerol ester as defined in the first aspect of the in vivo by a method as described above.

The inflammatory disease may be any disease as defined herein.

### Method of Diagnosis

In a further aspect, the present invention relates to a method for diagnosing an inflammatory disease in a subject or identifying a subject at risk of developing an inflammatory disease, comprising:

(a) determining a level of at least one oxygenated fatty acyl glycerol ester in a sample from the subject,
(b) comparing the level(s) of the oxygenated fatty acyl glycerol ester(s) in the sample to reference values;
wherein a lower level(s) of the oxygenated fatty acyl glycerol ester(s) in the sample compared to the reference levels is indicative of an inflammatory disease or the risk of developing an inflammatory disease.

### Determining a Level of at Least One Oxygenated Fatty Acyl Glycerol Ester

The levels of a oxygenated fatty acyl glycerol ester in the sample may be measured or determined by any suitable method. For example, mass spectroscopy (MS) may be used. Other spectroscopic methods, chromatographic methods, labeling techniques, or quantitative chemical methods may be used in alternative embodiments. The oxygenated fatty acyl glycerol ester levels in the sample may be measured by mass spectroscopy, in particular liquid chromatography tandem mass spectrometry (LC-MS/MS).

The oxygenated fatty acyl glycerol ester may be determined using a liquid chromatography (LC/MS/MS). For example, the level oxygenated fatty acyl glycerol ester may be determined using an LC/MS/MS method as described by Masoodi et al. (Leukemia (2014) 28, 1381-1387).

Typically the oxygenated fatty acyl glycerol ester level in the sample and the reference value are determined using the same analytical method.

### Sample

The present method comprises a step of determining the level of at least one oxygenated fatty acyl glycerol ester in a sample obtained from a subject. Thus the present method is typically practiced outside of the human or animal body, e.g. on a body fluid sample that was previously obtained from the subject to be tested. The sample may be derived from blood, i.e. the sample may comprise whole blood or a blood fraction. The sample may comprise blood plasma or serum.

Techniques for collecting blood samples and separating blood fractions are well known in the art. For instance, vena blood samples can be collected from patients using a needle and deposited into plastic tubes. The collection tubes may, for example, contain spray-coated silica and a polymer gel for serum separation. Serum can be separated by centrifugation at 1300 RCF for 10 min at room temperature and stored in small plastic tubes at −80° C.

The sample may be a serum, plasma, urine or adipose tissue biopsy sample.

### Comparison to Reference Values

The present method further comprises a step of comparing the level of at least oxygenated fatty acyl glycerol ester in the test sample to one or more reference or control values. Typically a specific reference value for each individual oxygenated fatty acyl glycerol ester determined in the method is used. The reference value may be a normal level of that oxygenated fatty acyl glycerol ester, e.g. a level of the oxygenated fatty acyl glycerol ester in the same sample type (e.g. serum or plasma) in a control subject. The control subject may, for example, be normal, healthy subject or an obese but non-diabetic subject. The reference value may, for example, be based on a mean or median level of the oxygenated fatty acyl glycerol ester in a control population of subjects, e.g. 5, 10, 100, 1000 or more control subjects (who may either be age- and/or gender-matched or unmatched to the test subject).

The extent of the difference between the subject's oxygenated fatty acyl glycerol ester biomarker levels and the corresponding reference values is also useful for determining which subjects would benefit most from certain interventions.

The level of the oxygenated fatty acyl glycerol ester in the test sample may be decreased by, for example, at least 1%, at least 5%, at least 10%, at least 20%, at least 30%, at least 50% or at least 100% compared to the reference value.

In some embodiments, the reference value is a value obtained previously from the same subject. This allows a direct comparison of the effects of a current lifestyle of the subject or a treatment strategy compared to a previous lifestyle or pre-treatment on oxygenated fatty acyl glycerol ester biomarker levels, so that improvements can be directly assessed.

The reference value may be determined using corresponding methods to the determination of oxygenated fatty acyl glycerol ester levels in the test sample, e.g. using one or more samples taken from control subjects. For instance, in some embodiments oxygenated fatty acyl glycerol ester levels in control samples may be determined in parallel assays to the test samples. Alternatively, in some embodiments reference values for the levels of individual oxygenated fatty acyl glycerol ester species in a particular sample type (e.g. serum or plasma) may already be available, for instance from published studies. Thus in some embodiments, the reference value may have been previously determined, or may be calculated or extrapolated, without having to perform a corresponding determination on a control sample with respect to each test sample obtained.

### Inflammatory Disease

The inflammatory disease may be any inflammatory disease as described herein. In one embodiment, the present method may be used may be used to predict the likelihood that an obese subject will develop TIID. As described above, although obesity is a major risk factor for the development of insulin resistance and potentially TIID, not all patients who are obese develop insulin resistance and TIID. The present inventors have surprisingly determined that levels of decreased levels of oxygenated fatty acyl glycerol esters are associated with the development of insulin resistance and TIID. Thus, in one embodiment of the present method, an obese subject may be predicted to have an increased likelihood of developing TIID if the level of a oxygenated fatty acyl glycerol ester in a sample derived from the subject is decreased by, for example, at least 1%, at least 5%, at least 10%, at least 20%, at least 30%, at least 50% or at least 100% compared to the reference value.

The present method may further comprise the step of treating a subject who is determined by the present method to have, or to be at risk of, an inflammatory disease by inducing or increasing production of at least one oxygenated fatty acyl glycerol ester by the method as defined herein.

The present invention also provides a oxygenated fatty acyl glycerol ester according to the first aspect of the invention for use in
i) regulating inflammatory cytokine signalling in a cell; or
ii) protecting a cell against apoptosis.

### Inflammatory Cytokine Signalling

Inflammation is mediated by a variety of inflammatory cytokines, which can be divided into two groups: those involved in acute inflammation and those responsible for chronic inflammatory responses. Inflammation, for example in response to tissue injury, is characterized in the acute phase by increased blood flow and vascular permeability along with the accumulation of fluid, leukocytes, and inflammatory mediators such as cytokines. In the subacute/chronic phase (hereafter referred to as the chronic phase), it is characterized by the development of specific humoral and cellular immune responses for example to the pathogen (s) present at the site of tissue injury. During both acute and chronic inflammatory processes, a variety of soluble factors are involved in leukocyte recruitment through increased expression of cellular adhesion molecules and chemoattraction. Many of these soluble mediators regulate the activation of the resident cells (such as fibroblasts, endothelial cells, tissue macrophages, and mast cells) and the newly recruited inflammatory cells (such as monocytes, lymphocytes, neutrophils, and eosinophils), and some of these mediators result in the systemic responses to the inflammatory process. Several cytokines play key roles in mediating acute inflammatory reactions, namely IL-1, TNF-α, IL-6, IL-11, IL-8 and other chemokines, GCSF, and GM-CSF. The cytokines known to mediate chronic inflammatory processes can be divided into those participating in humoral inflammation, such as IL-3, IL-4, IL-5, IL-6, IL-7, IL-9, IL-10, IL-13, and transforming growth factor-b (TGF-b), and those contributing to cellular inflammation such as IL-1, IL-2, IL-3, IL-4, IL-7, IL-9, IL-10, IL-12, interferons (IFNs), IFN-γ inducing factor (IGIF), TGF-β, and TNF-α and -β.

The oxygenated fatty acyl glycerol ester may regulate inflammatory cytokine signalling in a cell. In particular it may modulate the response of the cell to inflammatory cytokines such as IL-1β, TNFα and/or IFNγ.

The oxygenated fatty acyl glycerol ester may downregulate the NFkB signaling pathway activated by a cellular inflammatory response.

### Apoptosis

A cell initiates intracellular apoptotic signaling in response to a stress, such as heat, radiation, nutrient deprivation, viral infection or hypoxia. Before the actual process of cell death occurs, apoptotic signals must cause regulatory proteins to initiate the apoptosis pathway. Two main methods of regulation of this process have been identified: targeting mitochondria functionality, or directly transducing the signal via either the TNF path or the Fas path.

Endoplasmic reticulum stress, oxidative stress and inflammation are the main cause of beta cell dysfunction in diabetes. The present inventors have shown that the bioactive lipids of the present invention reduce the apoptotic signal in beta cells which had been treated with an inflammatory cytokine cocktail (Example 4). The bioactive lipids protected beta cells from apoptosis by reducing NFkB signaling pathway activated by cellular inflammatory response.

The invention will now be further described by way of Examples, which are meant to serve to assist one of ordinary skill in the art in carrying out the invention and are not intended in any way to limit the scope of the invention.

### Examples

### Example 1—Acute Stimulation of MIN6 Cells with Bioactive Lipid Fractions

MIN6 cells were cultured in complete DMEM medium at 70 to 80% confluent. To measure the acute effect of bioactive lipid fractions on glucose stimulated insulin secretion (GSIS), cells were starved in low glucose medium (Krebs Ringer Buffer Hepes or KRBH plus 2 mM glucose) for 2 hours before stimulation with 20 mM glucose in the presence of bioactive lipid fractions (1:50 dilution in KRBH 20 mM Glc) for 30 minutes. The effect of bioactive lipid on GSIS was compared to the control glucose plus vehicle (2% Ethanol). Insulin secretion was measured using the insulin ELISA kit (Mercodia).

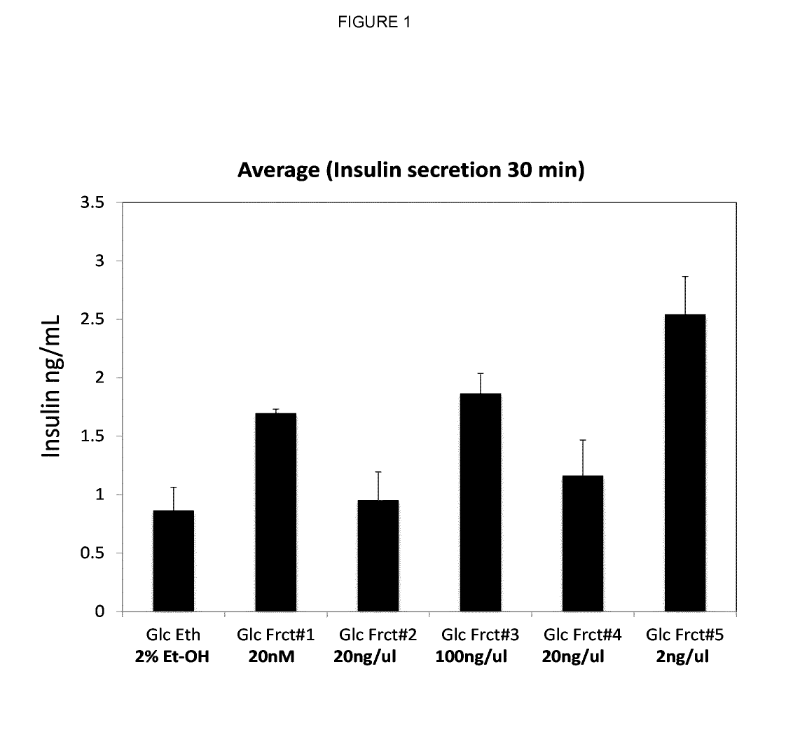

These data show that there is a synergistic effect of the bioactive lipids on GSIS (FIG. 1).

### Example 2—Effect of Chronic Treatment with Lipid Fractions on Beta Cell Function and Survival

To determine whether long-term treatment with bioactive lipids affected beta cell function and survival, the MIN6 beta cell line was treated with increasing concentrations of bioactive lipids (from 1:1000 to 1:20 dilution) in DMEM medium for 48 hrs (see Table 1). In the chronic treatment experiment, the bioactive lipids were removed from the medium at the end of the pretreatment and cellular function was assessed after glucose stimulation. Cell survival and proliferation was assessed by counting cell number compared to the baseline of vehicle treated cell normalized to 100%.

TABLE 1

| | Dilutions: | | | | | | | |
|---|---|---|---|---|---|---|---|---|
| | 1:1000 | | 1:100 | | 1:50 | | 1:20 | |
| Ethanol | 0.1% | | 1% | | 2% | | 5% | |
| Fraction1 | 1 | nM | 10 | nM | 20 | nM | 50 | nM |
| (1 uM) | | | | | | | | |
| Fraction 2 | 1 | ng/ul | 10 | ng/ul | 20 | ng/ul | 50 | ng/ul |
| (1 ug/ul) | | | | | | | | |
| Fraction 3 | 5 | ng/ul | 50 | ng/ul | 100 | ng/ul | 250 | ng/ul |
| (5 ug/ul) | | | | | | | | |
| Fraction 4 | 1 | ng/ul | 10 | ng/ul | 20 | ng/ul | 50 | ng/ul |
| (1 ug/ul) | | | | | | | | |
| Fraction 5 | 0.1 | ng/ul | 1 | ng/ul | 2 | ng/ul | 5 | ng/ul |
| (0.1 ug/ul) | | | | | | | | |

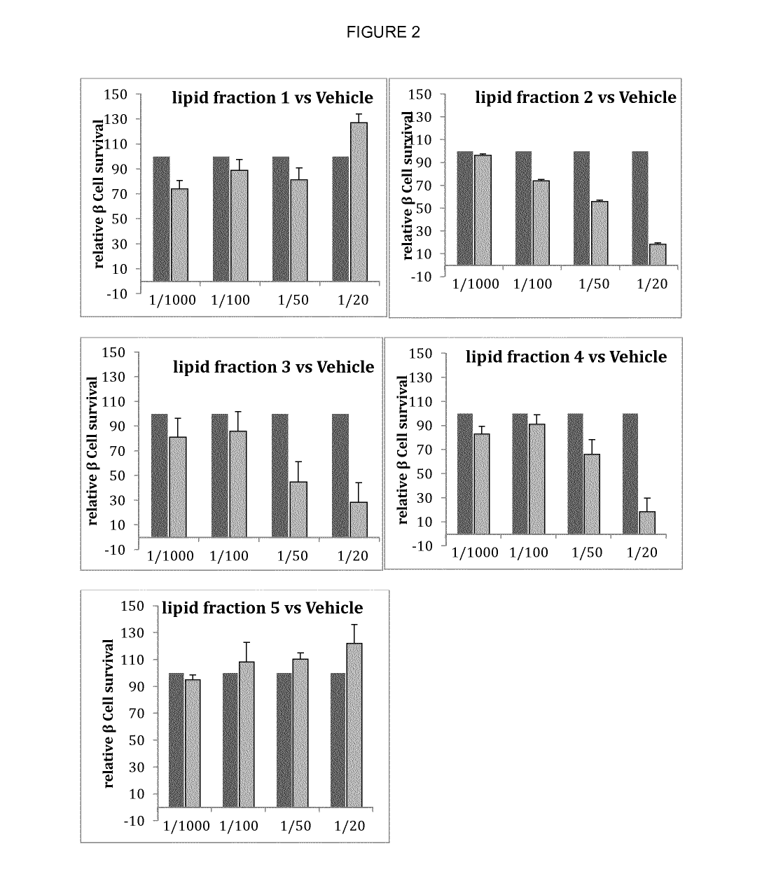

At the highest concentration of bioactive lipid only fractions 1 and 5 show an effect in cell survival (FIG. 2).

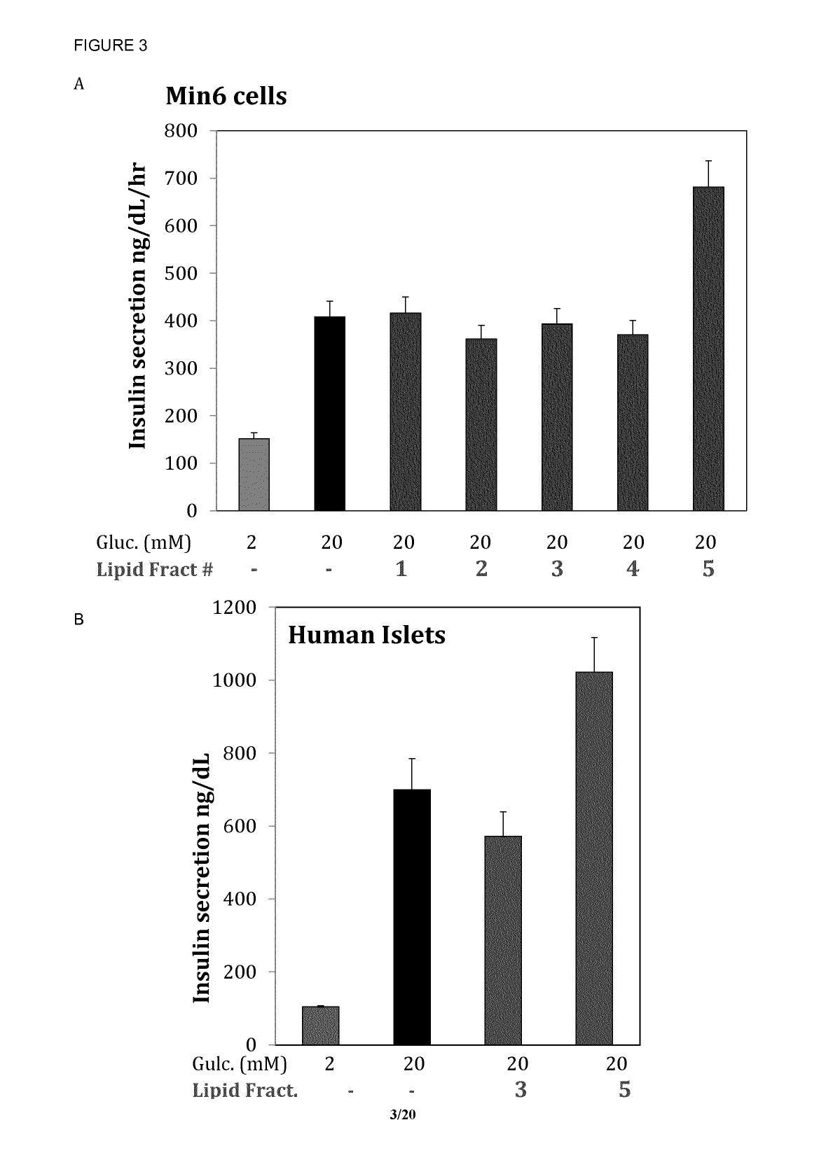

To determine the long-term effect of bioactive lipids on beta cell function, MIN6 cells were treated with bioactive lipids at a concentration close to physiological ranges (1:1000 dilution) for 72 hours. At the end of the treatment, beta cell function was assessed by measuring GSIS (FIG. 3A). The bioactive lipid fraction 5 substantially improved beta cells function by doubling the capacity of MIN6 cells to secrete insulin in response to glucose stimulation.

Fractions 3 and 5 were further tested in primary human islets from a healthy donor. Fraction 5 significantly increased insulin secretion in response to glucose stimulation from already healthy islets (FIG. 3B).

### Example 3—Further Determination of the Effect of Bioactive Lipids on Beta Cell Function

Isolation of pure bioactive lipid species from lipid fraction 5 was performed by further fractionation using liquid chromatography. Five sub-fractions were isolated and tested to determine if they acutely stimulated insulin secretion in the presence of glucose.

Insulin secretion was measured in MIN6 cells under starving condition (2 mM glucose) or after stimulation with 20 mM glucose or 20 mM glucose plus bioactive lipids at a 1:100 dilution for 15 minutes. Insulin secretion was measured by ELISA.

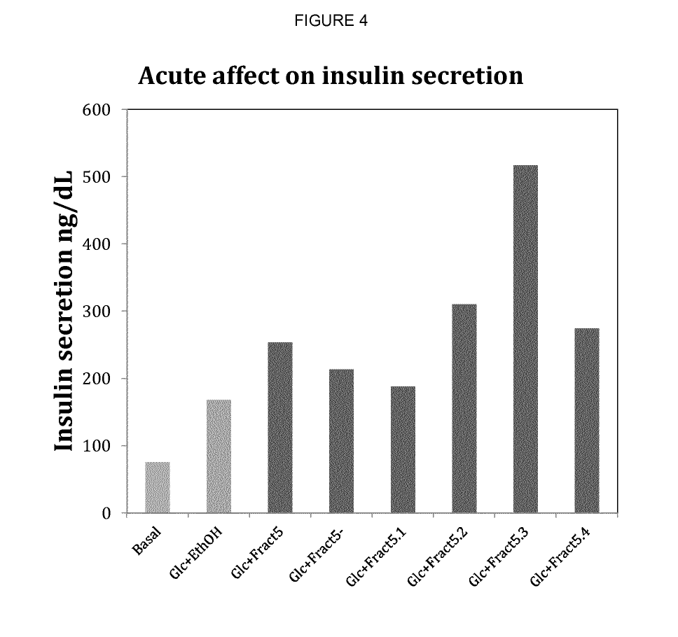

Fraction 5 synergistically increased insulin secretion in the presence of glucose, but the sub-fraction 5.3 augmented insulin secretion nearly three fold above glucose alone (FIG. 4).

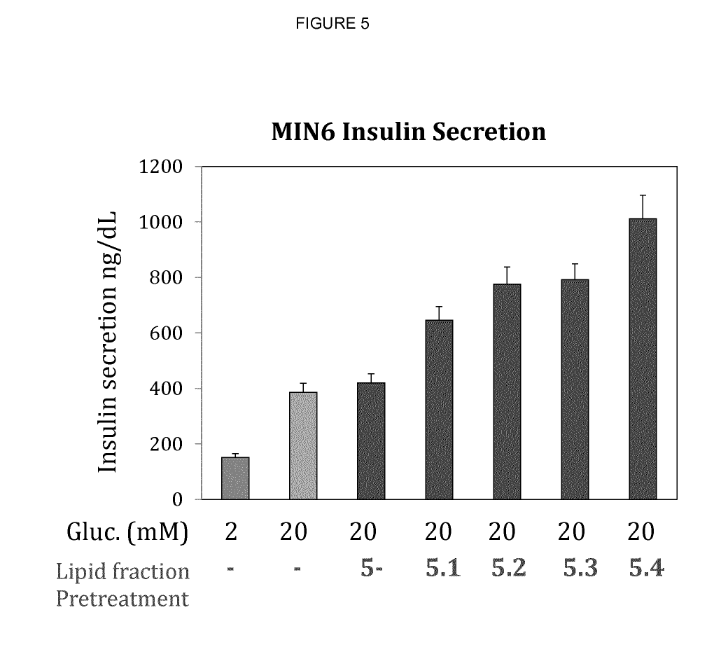

To determine the effect of long-term treatment, MIN6 cells were treated with the enriched bioactive lipid sub-fractions for 72 hrs in a 1:1000 dilution before performing GSIS (FIG. 5). All the sub-fractions substantially improved beta cell function; however the sub-fraction 5.4 is a more potent modulator of beta cell function.

### Example 4—Cytoprotective Effect of Bioactive Lipids

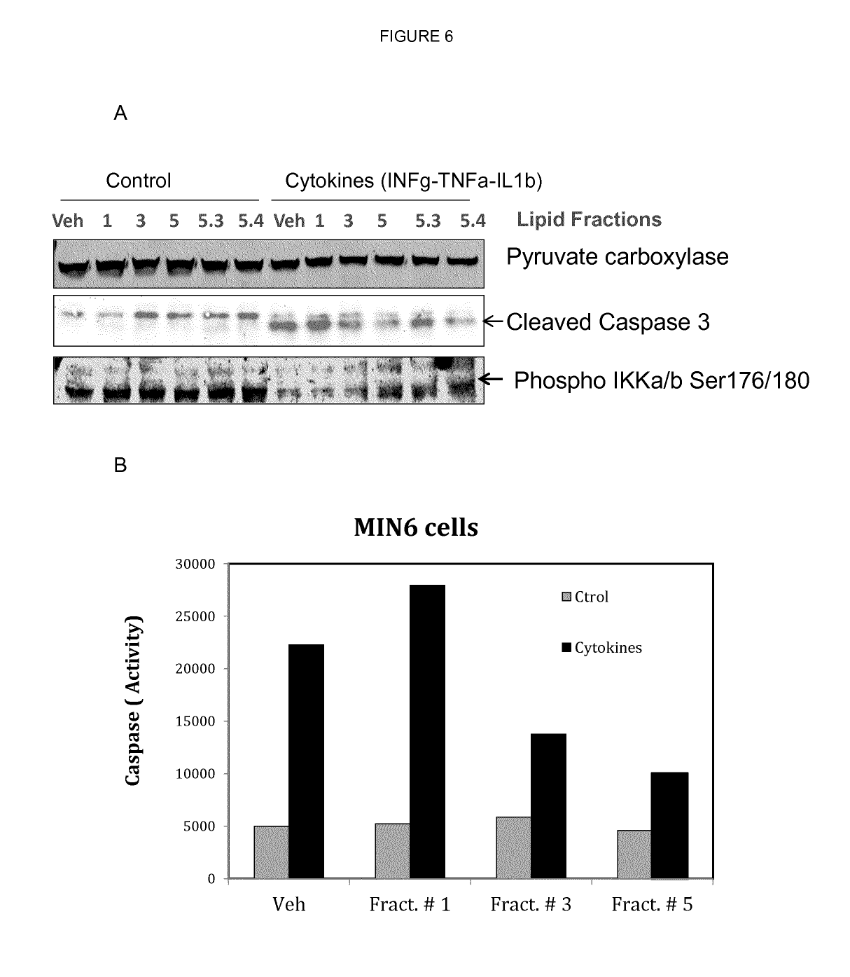

Endoplasmic reticulum stress, oxidative stress and inflammation are the main cause of beta cell dysfunction in diabetes. To determine if the bioactive lipid fractions isolated play a role in beta cell death, beta cells were treated with an inflammatory cytokine cocktail (50 U/mL IL1β, 100 U/mL TNFα and 100 U/mL IFNγ) for 48 hrs in the presence or absence of bioactive lipid fractions (1:100 dilution). After treatment, caspase 8 activity (an early marker of apoptosis) is measured from crude cell extract. Both fraction 3 and fraction 5 reduced the apoptotic signal, indicating that both fraction 3 and fraction 5 have cytoprotective properties (FIG. 6A).

The cytoprotective properties of sub-fraction 5.3 and 5.4 was further assessed in comparison to fractions 1, 3 and 5 in MIN6 cells after cytokine treatment as described above. Both fraction 5 and sub-fraction 5.4 significantly reduced cleaved caspase 3 (apoptosis) by reducing NFkB signaling pathway activated by cellular inflammatory response (FIG. 6B).

### Example 5—Physiological Relevance of Bioactive Lipids in Beta Cell Dysfunction

The effect of bioactive lipid sub-fraction 5.4 on the function of primary islets isolated from GK (Gata Kakizaki) rats, a type 2 diabetes models very similar to human type 2 diabetes, were further investigated. The adult GK rats are characterized by marked inflammation, islet cell fibrosis and reduced beta cell function. To determine if bioactive lipid fraction 5.4 rescued the islet dysfunction in GK rat, isolated islets were treated with either vehicle or 5.4 fraction for 72 hours before assessing islet function after stimulation with a secretagogue cocktail composed of 20 mM glucose, 1× amino acid and 0.1 μM Ex-4, a GLP1 isoform for 1 hr.

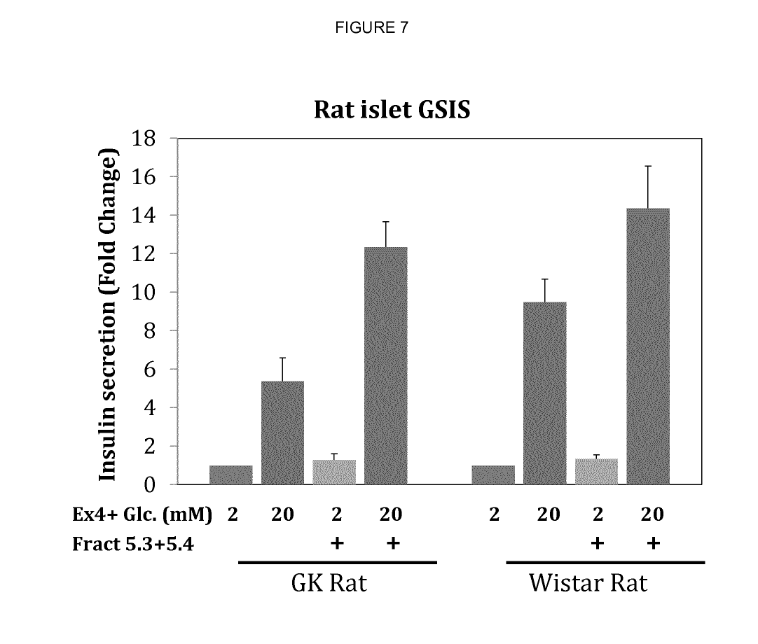

These data indicate that Fraction 5.4 is capable of rescuing insulin secretion capacity of GK rats to levels comparable to the normal Wistar rat control (FIG. 7).

### Example 6—Role of Bioactive Lipids in Enteroendocrine Cell Secretion of Glucagon-Like Peptide 1 (GLP1)

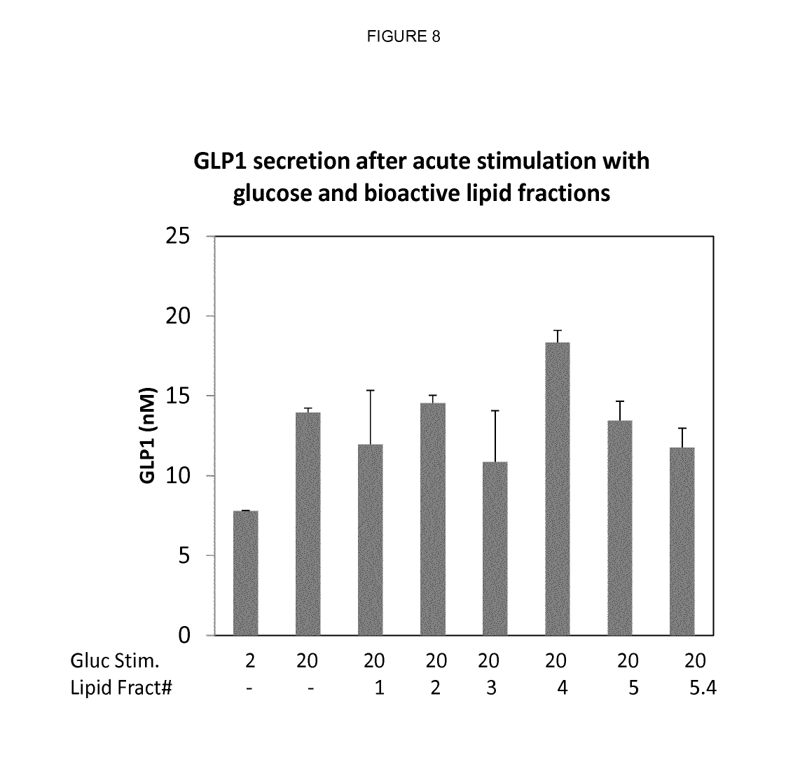

The enteroendocrine L cell line (NCI-H716) was acutely stimulated with low (2 mM) and high (20 mM) concentrations of glucose in the presence or absence of bioactive lipid fractions. These data indicate that fraction 4 provided the most significant synergy with stimulatory glucose to increase GLP1 secretion (FIG. 8).

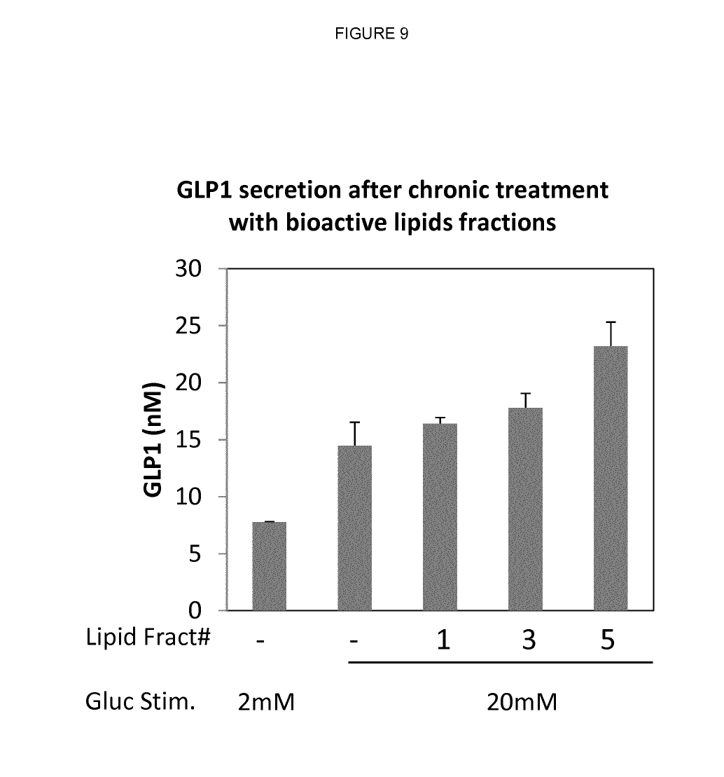

In order to determine the long-term effect of bioactive lipids in enteroendocrine L cell function, the NCI-H716 cell line was treated with the bioactive lipids for 72 hrs. Fraction 5 pretreatment substantially increased GLP1 secretion after stimulation with 20 mM Glucose (FIG. 9).

### Example 7—Bioactive Lipids Regulate Cellular Stress Genes

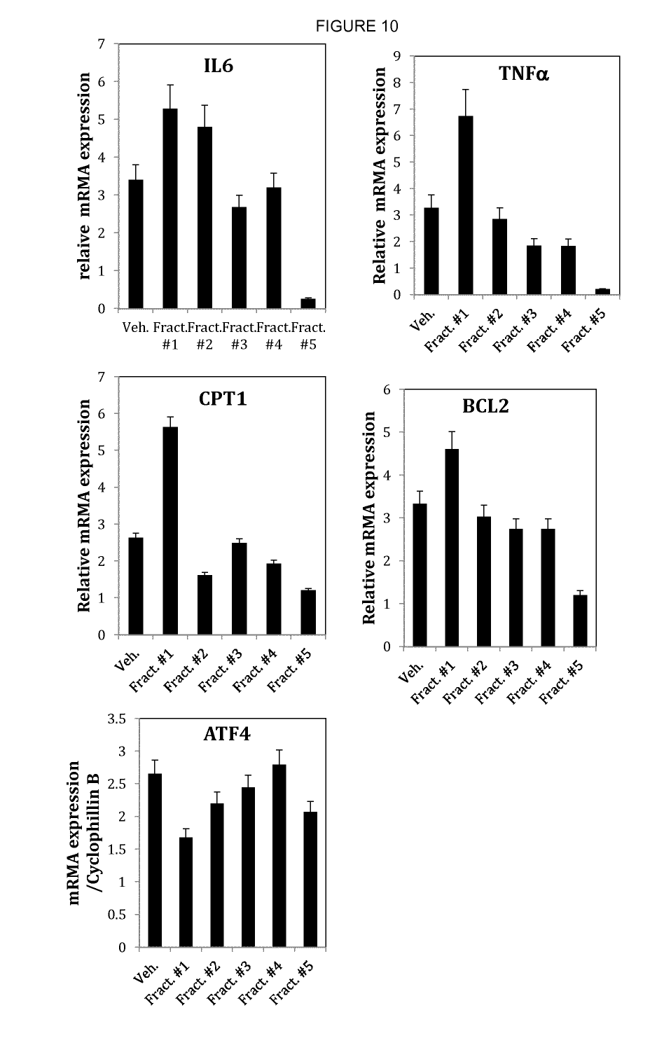

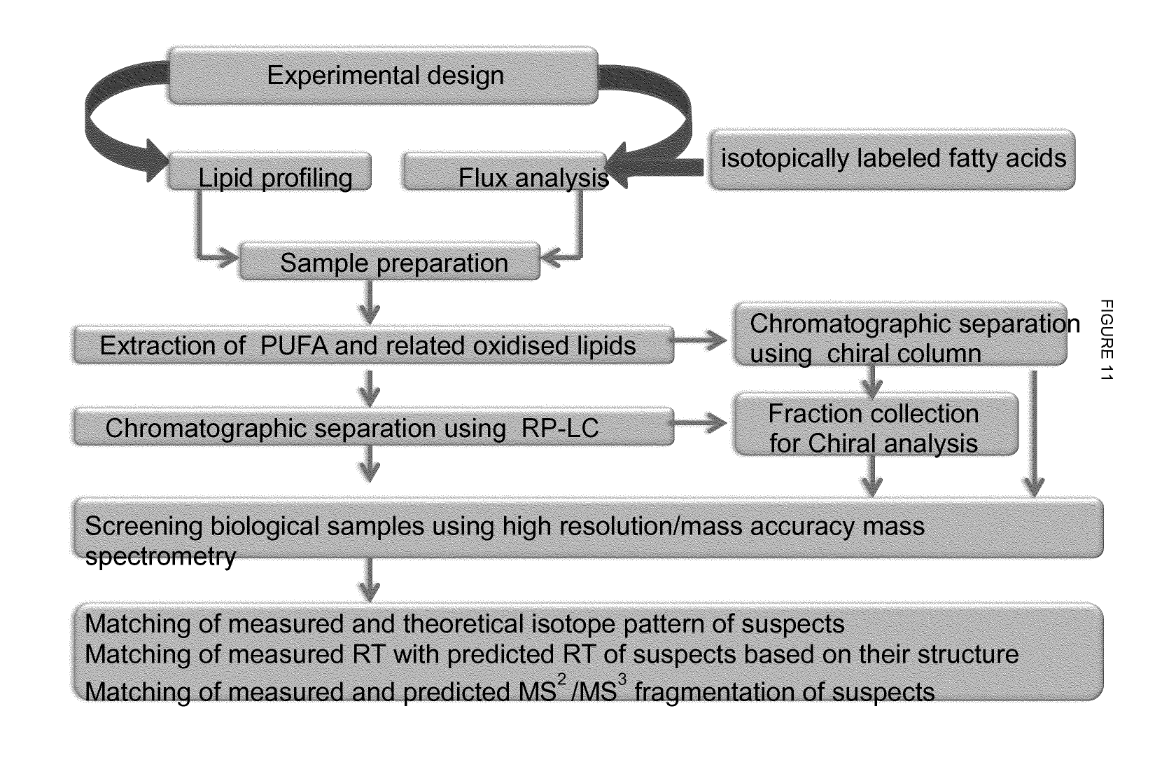

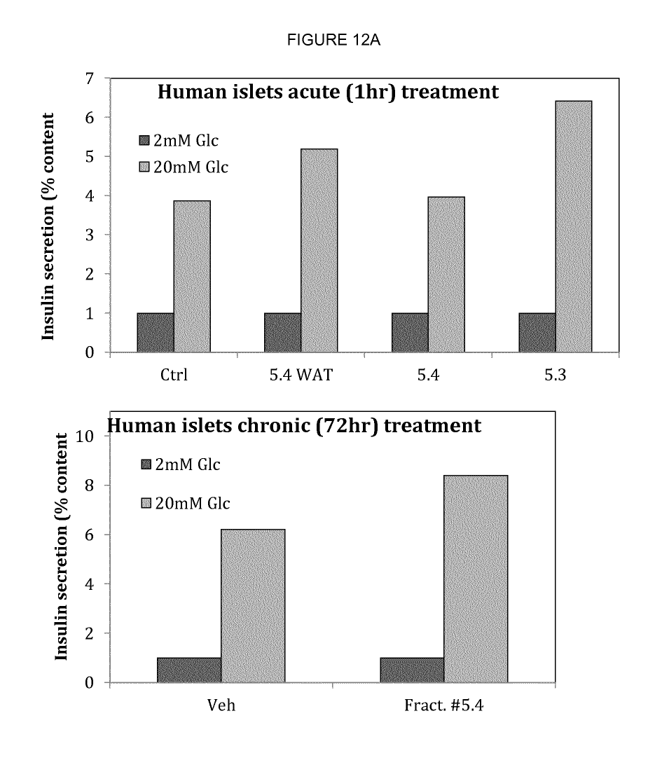

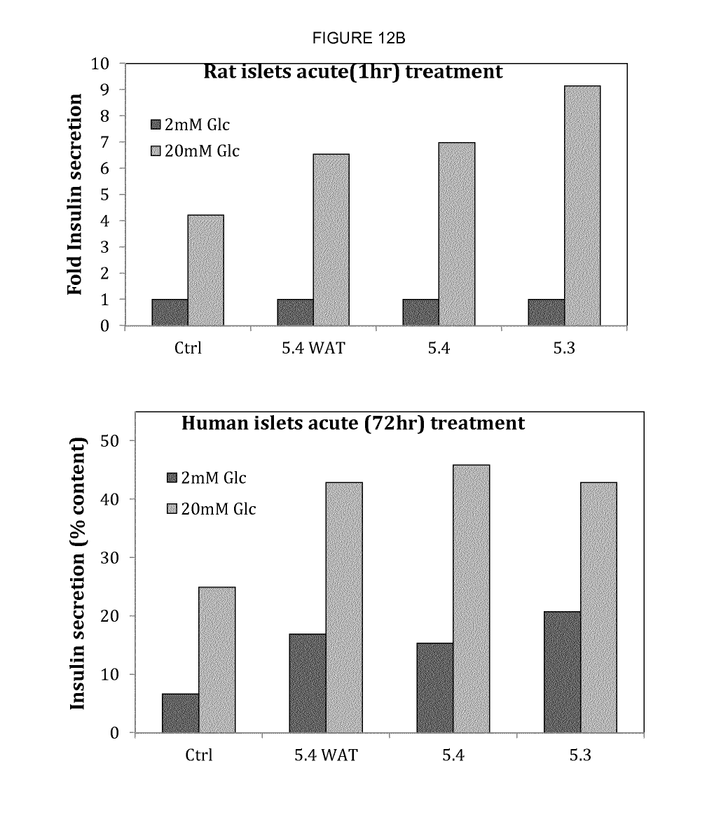

Bioactive lipid fractions are capable of reducing the expression of cellular stress genes associated with inflammation and endoplasmic reticulum stress (FIG. 10). In particular, Fraction 5 worked best.

### Example 8—Identification of Bioactive Lipids

Chromatographic analyses were performed as described in Masoodi et al. (Leukemia (2014) 28, 1381-1387). Eicosanoids and related metabolites were separated on a C18 reversed-phase (RP) LC column (Phenomenex Luna, 3 μm particles, 150×2 mm) and fatty acyl ethanolamides/glycerol esters were separated on (Phenomenex Kinetex-XB-C18, 2.6 μm particles, 100×2 mm) using a gradient (A: 10 mM ammonium acetate+ 0.1% formic acid; B: ΔCN: H2O: formic acid (90:10:0.1)+10 mM ammonium acetate at 0.5 mL/min. Starting conditions consisted of 35% B and were maintained for 2 min. The gradient then increased to 55% B over 1 min followed by an increase to 95% B over 7 min, maintained for 2 min and finally returned to the initial conditions for 2 min to allow equilibration.

Mass spectrometry analyses were carried out using an LTQ Elite linear ion trap (LIT)-orbitrap. The ion spray voltage was adjusted to 4000 V. Resolving powers of 60000 in full scan mode and 15000 in MS/MS mode were used. For automated data processing, data acquisition files were converted to open *.mzXML file standard and analyses were carried out using the open-source Bioconductor packages XCMS (version 1.22.1)2 as well as additional R packages developed in-house. Peak detection was carried out on centroided peaks and sample-dependent mass-recalibration was carried out using internal mass standards as well as common intact lipids. Peaks were grouped across the whole sample set with a mass tolerance of 5 ppm. Peak de-isotoping was carried out using a hierarchical, correlation based approach developed in-house with a maximum mass deviation of 3 ppm.

Representative bioactive lipids identified using this method were: 9α, 15S-dihydroxy-11-oxo-5Z,13E-prostadienoic acid 1-glyceryl ester or 9α,15S-dihydroxy-11-oxo-prosta-5Z,13E-dien-1-oic acid 1-glyceryl ester or prostaglandin D2 glycerol ester (from fraction 5.3)

11-oxo-5Z,9,12E,4E-prostatetraenoic acid-2-glycerol ester or 11-oxo-prosta-5Z,9,12E,14E-tetraen-1-oic acid, 2-glycerol ester or 15-deoxy-Δ12,14-PGJ2-2-glycerol ester (from fraction 5.4)

Purified bioactive lipids were then used for subsequent in vitro testing.

### Example 9—Comparison of the Functional Effects of Isolated Fraction 5.4 with Synthetic Pure Fractions 5.4 and 5.3

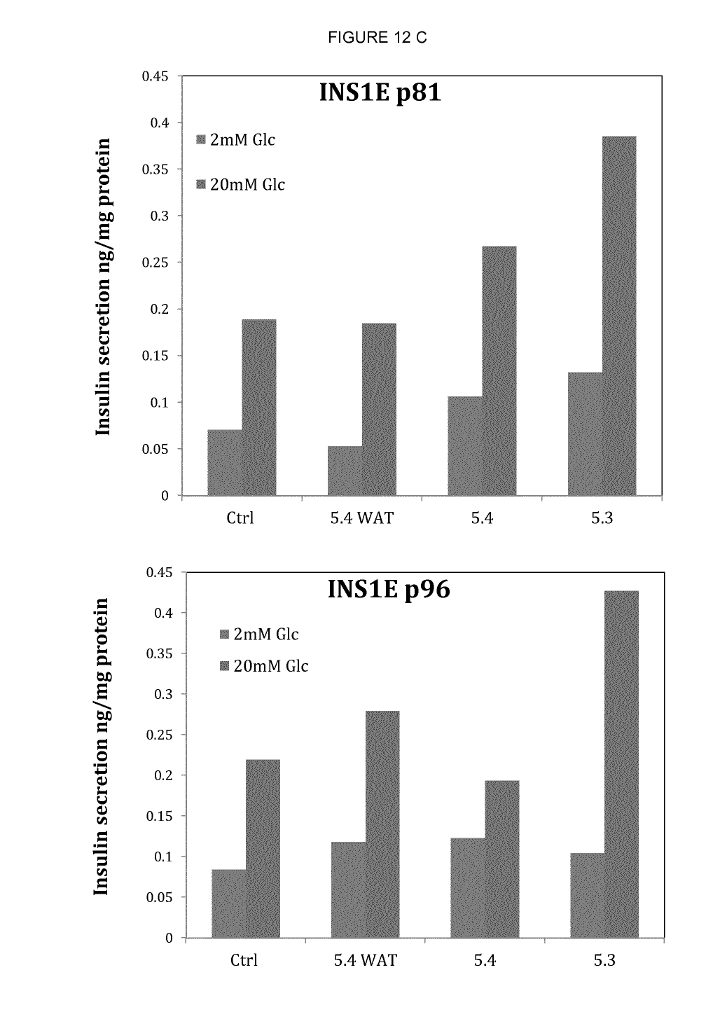

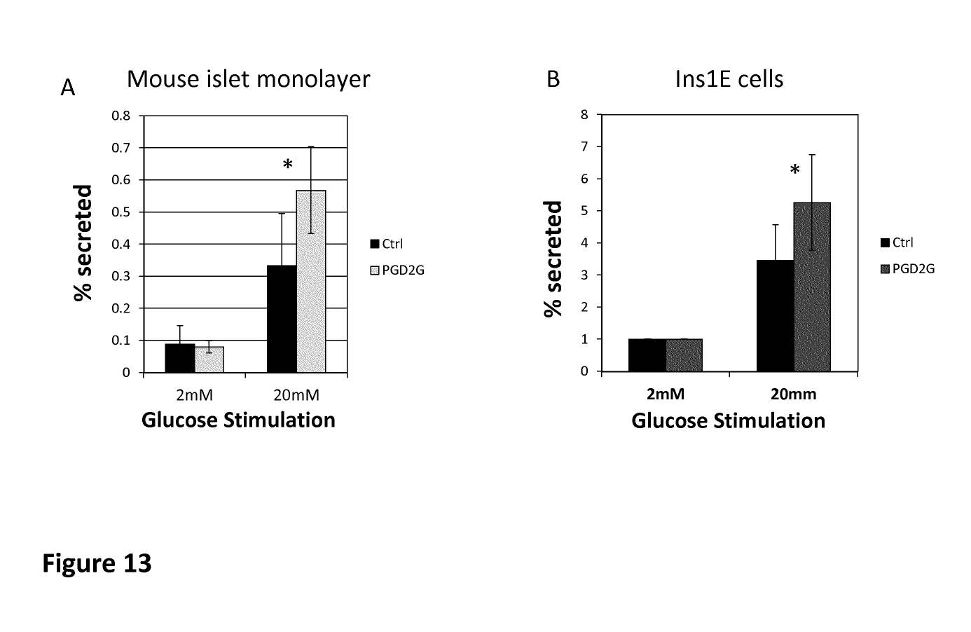

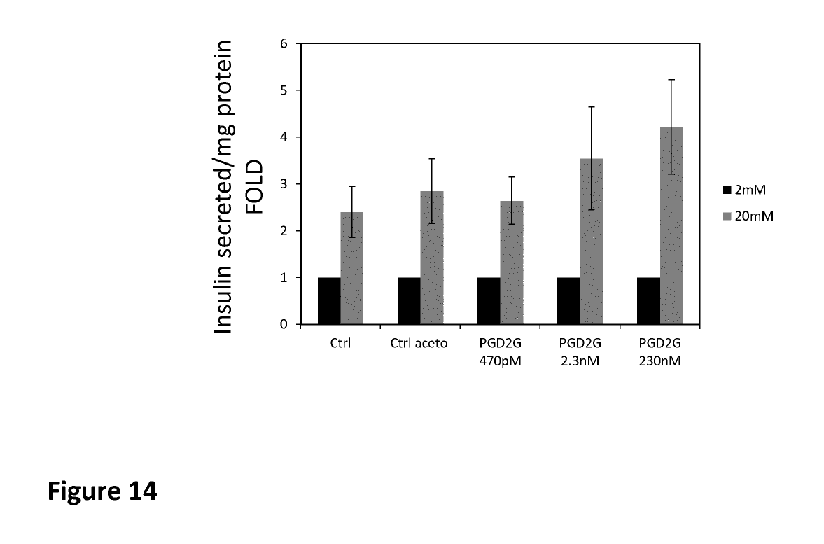

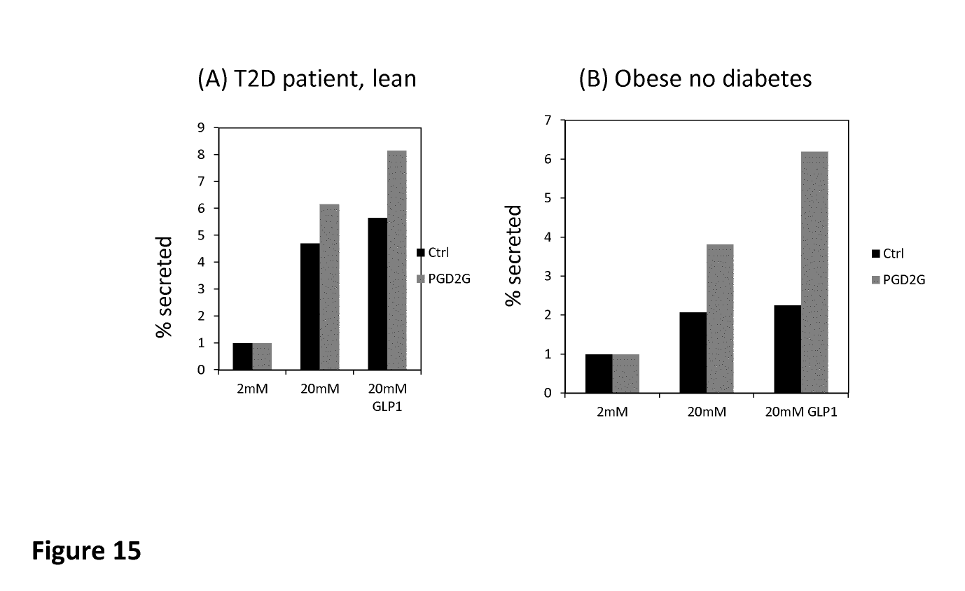

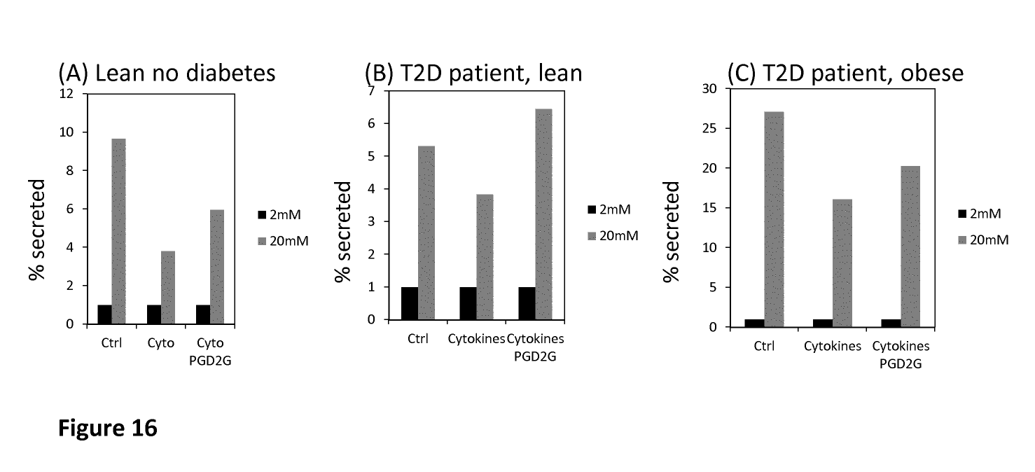

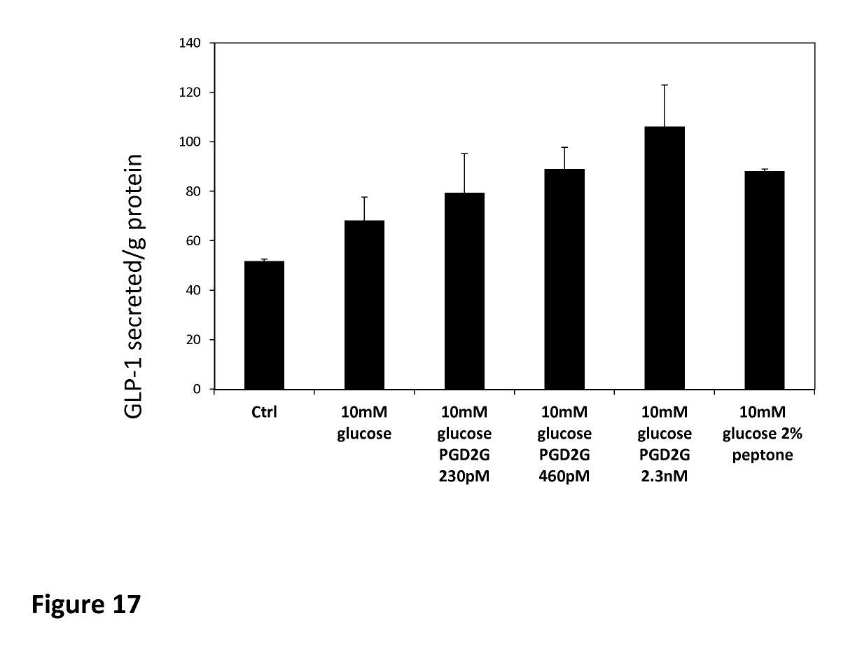

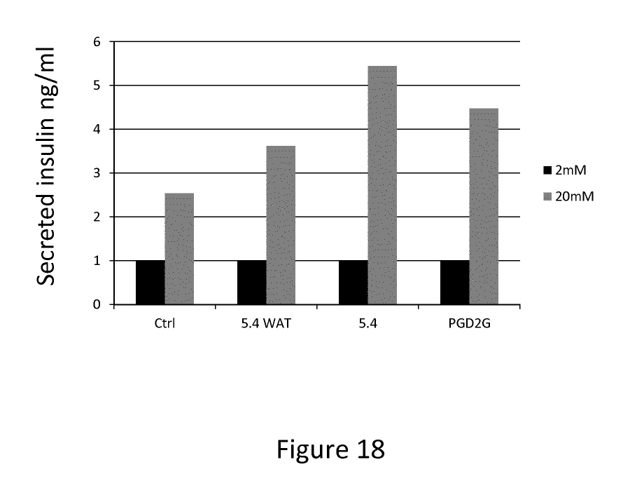

Insulin secretion was measured in primary rat and human islets after acute treatment (1 hour) or chronic treatment (16 or 72 hours, 1:500 dilution) with the bioactive lipid and glucose (FIG. 12).

Fraction 5.4WAT was purified from adipose/brain tissue. Fractions 5.4 and 5.3 are synthetic pure fractions (10 μg/μl stock). The concentration of bioactive lipid was 20 pg/μl. The control was ethanol. As shown in FIG. 12, the synthetic compound was found to have similar chemical and biological effects as fraction 5.3/5.4.

1. An oxygenated fatty acyl glycerol ester for use in treating and/or preventing an inflammatory disease in a subject.

2. An oxygenated fatty acyl glycerol ester for use according to claim 1 which oxygenated fatty acyl glycerol ester is an oxygenated arachidonyl glycerol ester.

3. An oxygenated fatty acyl glycerol ester for use according to claim 1 which oxygenated fatty acyl glycerol ester is a prostaglandin glycerol ester.

4. An oxygenated fatty acyl glycerol ester for use according to claim 1 which oxygenated fatty acyl glycerol ester is a prostatetraenoic acid glycerol ester.

5. A prostatetraenoic acid glycerol ester for use according to claim 4 wherein the prostatetraenoic acid glycerol ester is selected from the following group:
11-oxo-5Z,9, 12E, 14E-prostatetraenoic acid-1 glycerol ester;
9, 15S-dihydroxy-11-oxo-5Z, 13E-prostadienoic acid, 1-glyceryl ester;
11-oxo-5Z,9,12E,4E-prostatetraenoic acid-2-glycerol ester;
11-oxo-15S-hydroxy-5Z,9Z,13E-prostatrienoic acid-1 glycerol ester; and
9, 15S-dihydroxy-11-oxo-5Z,13E-prostadienoic acid, 2-glyceryl ester.

6. A prostatetraenoic acid glycerol ester for use according to claim 5 wherein the prostatetraenoic acid glycerol ester is 11-oxo-5Z,9,12E,14E-prostatetraenoic acid-1 glycerol ester; 9, 15S-dihydroxy-11-oxo-5Z,13E-prostadienoic acid, 1-glyceryl ester; 11-oxo-5Z,9,12E,14E-prostatetraenoic acid-2-glycerol ester or 9, 15S-dihydroxy-11-oxo-5Z,13E-prostadienoic acid 2-glyceryl ester.

7. A composition comprising one or more oxygenated fatty acyl glycerol esters as defined in claim 1 for use in treating and/or preventing an inflammatory disease in a subject.

8. An oxygenated fatty acyl glycerol ester or composition for use according to claim 1 wherein the inflammatory disease is selected from the following group: Type II diabetes, insulin resistance, obesity and metabolic diseases.

9. An oxygenated fatty acyl glycerol ester or composition for use according to claim 8 wherein the inflammatory disease is Type II diabetes.

10. An oxygenated fatty acyl glycerol ester or composition for use according to claim 9 for preventing or delaying the onset of Type II diabetes in an obese subject.

11. An oxygenated fatty acyl glycerol ester or composition for use according to claim 1 for modulating insulin secretion in a subject.

12. An oxygenated fatty acyl glycerol ester or composition for use according to claim 1 wherein the prostaglandin glycerol ester acts on a cell selected from the following group: a pancreatic cell, an enteroendocrine cell, an epithelial cell, a liver cell, an adipocyte, or a neural cell.

13. An oxygenated fatty acyl glycerol ester or composition for use according to claim 12 wherein the cell is a pancreatic beta cell.

14. An oxygenated fatty acyl glycerol ester or composition for use according to claim 13 wherein the use increases the level of insulin produced by the beta cell.

15. An oxygenated fatty acyl glycerol ester or composition for use according to claim 13, where the use prevents or reduces apoptosis of pancreatic beta cells.

16. An oxygenated fatty acyl glycerol ester or composition for use according to claim 12 wherein the cell is an L cell.

17. An oxygenated fatty acyl glycerol ester or composition for use according to claim 12 wherein the cell is an astrocyte or a neuron.

18. An oxygenated fatty acyl glycerol ester or composition for use according to claim 1 wherein the use reduces inflammation in liver and/or adipose tissues.

19. A method for inducing or increasing production of at least one oxygenated fatty acyl glycerol ester as defined in claim 1 in vivo.

20. A method according to claim 19 wherein the oxygenated fatty acyl glycerol ester is increased in a liver cell or white adipose tissue.

21. A method according to claim 19 which comprises the step of:
(a) administering a precursor selected from the following group arachidonyl glyercol (AG), diacylglycerol (1,2-DAG) and/or triacylglycerol (TAG) to a subject.
(b) inducing or increasing the expression or activity of an enzyme selected from the following group Phospholipase C (PLC), Diacylglycerol lipase (DAGL), Phospholipase A2 (PLA₂), N-acetyltransferase 2 (NAT), N-acyl phosphatidylethanolamine-specific phospholipase D (NATE-PLD), Cyclooxygenase-2 (COX-2), prostaglandin F synthase (PGFS), prostaglandin E synthase (PROSTAGLANDIN GLYCEROL ESTER S), prostaglandin I synthase (PGIS), prostaglandin D synthase (PGDS) and/or thromboxane A(2) synthase (TXAS) in a subject.

22. A method for treating and/or preventing an inflammatory disease in a subject which comprises the step of administering a oxygenated fatty acyl glycerol ester as defined in claim 1 to a subject or inducing or increasing production of at least one prostaglandin glycerol ester as defined in claim in vivo by a method according to claim 19.

23. A method according to claim 22 wherein the inflammatory disease is selected from the following group: Type II diabetes, insulin resistance, obesity and metabolic diseases.

24. A method according to claim 23 wherein the inflammatory disease is Type II diabetes.

25. A method according to claim 23 for preventing or delaying the onset of Type II diabetes in an obese subject.

26. A method according to claim 23 for modulating insulin secretion in a subject.

27. A method for diagnosing an inflammatory disease in a subject or identifying a subject at risk of developing an inflammatory disease, comprising:
(a) determining a level of at least one oxygenated fatty acyl glycerol ester(s) in a sample from the subject,
(b) comparing the level(s) of the oxygenated fatty acyl glycerol ester(s) in the sample to reference values;
wherein a lower level(s) of the oxygenated fatty acyl glycerol ester(s) in the sample compared to the reference levels is indicative of an inflammatory disease or the risk of developing an inflammatory disease.

28. A method according to claim 27 wherein the oxygenated fatty acyl glycerol ester is an oxygenated arachidonyl glycerol ester.

29. A method according to claim 27 wherein the oxygenated fatty acyl glycerol ester is a prostaglandin glycerol ester.

30. A method according to claim 28 wherein the oxygenated fatty acyl glycerol is a prostatetraenoic acid glycerol ester.

31. A method according to claim 30 wherein the prostatetraenoic acid glycerol ester is selected the following group:
11-oxo-5Z,9,12E,14E-prostatetraenoic acid-1 glycerol ester;
9,15S-dihydroxy-11-oxo-5Z,13E-prostadienoic acid, 1-glyceryl ester;
11-oxo-5Z,9,12E,4E-prostatetraenoic acid-2-glycerol ester;
11-oxo-15S-hydroxy-5Z,9Z,13E-prostatrienoic acid-1 glycerol ester; or
9,15S-dihydroxy-11-oxo-5Z,13E-prostadienoic acid, 2-glyceryl ester.

32. A method according to claim 27 wherein the sample is a serum, plasma, urine sample or an adipose tissue biopsy.

33. A method according to claim 27 wherein the inflammatory disease is selected from the following group: Type II diabetes, insulin resistance, obesity and metabolic diseases.

34. A method according to claim 33 wherein the subject is obese and the method is to predict the likelihood of developing Type II diabetes.

35. A method according to claim 27, further comprising the step of inducing or increasing production of at least one oxygenated fatty acyl glycerol by the method as defined in claim 19.

36. A prostaglandin glycerol ester as defined in claim 1 for use in
i) regulating inflammatory cytokine signalling in a cell; or
ii) protecting a cell against apoptosis.
